(19) United States
(12) Patent Application Publication
(10) Pub. No.: US 2021/0038736 A1
(43) Pub. Date: Feb. 11, 2021

(54) NANOPARTICLES INCLUDING A GLATIRAMOID USEFUL IN POLYNUCLEOTIDE DELIVERY

(71) Applicants: King AbdulAziz University, Jeddah (SA); University of Kansas, Lawrence, KS (US)

(72) Inventors: Cory J. BERKLAND, Lawrence, KS (US); Nabil Abdulhafiz ALHAKAMY, Jeddah (SA)

(73) Assignees: King AbdulAziz University, Jeddah (SA); University of Kansas, Lawrence, KS (US)

(21) Appl. No.: 16/914,422

(22) Filed: Jun. 28, 2020

Related U.S. Application Data
(63) Continuation of application No. PCT/US2018/067978, filed on Dec. 28, 2018.
(60) Provisional application No. 62/612,110, filed on Dec. 29, 2017.

(51) Int. Cl. A61K 47/62; A61K 47/69; A61K 38/19; C12N 15/117

(52) U.S. Cl. CPC A61K 47/62; A61K 47/6931; C12N 2310/3513; C12N 15/117; C12N 2310/17; A61K 38/191

(57) ABSTRACT

The present technology is directed to composition that may be formulated for parenteral administration, where the position includes a plurality of nanoparticles and optionally a pharmaceutically acceptable carrier. Each nanoparticle of the plurality includes a glatiramoid and one or more of a polyinosine-polycytidylic acid (Poly(LC)), a plasmid DNA (pDNA), a CpG oligodeoxynucleotide (CpG ODN), or a combination of any two or more thereof.

### CROSS-REFERENCE TO RELATED APPLICATIONS

This application is a Continuation Application of International Application No. PCT/US2018/067978, filed Dec. 28, 2018, which claims the benefit of and priority to U.S. Provisional Application No. 62/612,110, filed Dec. 29, 2017, the entirety of each of which is hereby incorporated by reference for any and all purposes.

### SEQUENCE LISTING

The instant application contains a Sequence Listing which has been submitted electronically in ASCII format and is hereby incorporated by reference in its entirety. Said ASCII copy, created on Sep. 29, 2020, is named 104434-0212 SL.txt and is 1,888 bytes in size.

### FIELD

The present technology is directed to nanoparticle compositions useful for the delivery of polynucleotides.

### SUMMARY

In an aspect, a composition is provided that includes a plurality of nanoparticles, optionally where the compostion is formulated for parenteral administration. Each nanoparticle of the plurality includes a glatiramoid as well as one or more of a polyinosine-polycytidylic acid (Poly(I:C)), a plasmid DNA (pDNA), a CpG oligodeoxynucleotide (CpG ODN), or a combination of any two or more thereof Also provided are medicaments directed to such compositions as well as methods of use of such compositions and/or medicaments.

### DESCRIPTION OF THE DRAWINGS

FIGS. 1A-D provide the results of agarose gel electrophoresis studies of GA-pDNA nanoparticles (FIG. 1A), K₁₀₀-pDNA nanoparticles (FIG. 1B), and K₉-pDNA nanoparticles (FIG. 1C) at N/P ratios of 1, 2, 3, 4, 5, 10, 15, 30, and 60. FIG. 1D provides such results for GA-pDNA, K₁₀₀-pDNA, and K₉-pDNA nanoparticles at N/P ratio of 0.5. Red Star refers to where the nanoparticles were able to immobilize pDNA completely (“M” refers to marker).

FIGS. 2A-F provides the results of the evaluation of the particle sizes (effective diameters) of GA-pDNA, K₁₀₀-pDNA, K₉-pDNA, PEI-pDNA nanoparticles in the presence or absence of CaCl₂. The particle size of the nanoparticles (N/P ratios of 5, 10, 30, and 60) were determined by DLS in the presence of various concentrations of CaCl₂ (0 and 38 mmol/L) in GA-pDNA nanoparticles in nuclease-free water (NFW) (FIG. 2A), or in GA-pDNA nanoparticles in serum-free F-12 media (SFM) (FIG. 2B), FIG. 2C provides GA-pDNA, K₁₀₀-pDNA, K₉-pDNA, and PEI-pDNA nanoparticles in nuclease-free water (NFW) at N/P ratio of 10, and FIG. 2D provides GA-pDNA, K₁₀₀-pDNA, K₉-pDNA, and PEI-pDNA nanoparticles in serum-free F-12 media (SFM) at N/P ratio of 10. Evaluation of zeta potentials of GA-pDNA, K₁₀₀-pDNA, K₉-pDNA, and PEI-pDNA nanoparticles in the absence and presence of CaCl₂, where FIG. 2E provides GA-pDNA nanoparticles at N/P ratios of 1, 5, 10, 30, 60, and FIG. 2F provides GA-pDNA, K₁₀₀-pDNA, K₉-pDNA, and PEI-pDNA nanoparticles at N/P ratio of 10. Results are presented as mean ±SD (n=3).

FIGS. 3A-D provide SYBR Green fluorescence of GA-pDNA (FIG. 3A), K₁₀₀-pDNA (FIG. 3B), and K₉-pDNA nanoparticles (FIG. 3C) at N/P ratio of 10 with different dextran sulfate concentrations (0, 0.01, 0.1, and 1 mg/ml), and FIG. 3D provides GA-pDNA nanoparticles at N/P ratios of 1, 5, 10, 30, and 60 the presence or absence of 0.1 mg/ml of dextran sulfate.

FIGS. 4A-C provide the transfection efficiency of GA-pDNA (FIG. 4A), K₁₀₀-pDNA (FIG. 4B), and K₉-pDNA (FIG. 4C) nanoparticles in the absence of CaCl₂ (0 mmol/L) at N/P ratios of 5, 10, 30, and 60 (in A549 cells). PEI-pDNA nanoparticles (N/P ratio of 10) were used as a positive control. RLUs refers to relative light units. FIGS. 4D-F provide the transfection efficiency of GA-pDNA (FIG. 4D), K₁₀₀-pDNA (FIG. 4E), and K₉-pDNA (FIG. 4F) nanoparticles in the presence of 38 mmol/L CaCl₂ at N/P ratios of 5, 10, 30, and 60 (in A549 cells). PEI-pDNA nanoparticles (N/P ratio of 10) were used as a positive control. RLUs refers to relative light units. Results are presented as mean ±SD (n=4) ( [symbol] <0.0001 comparison to pDNA) (***=P<0.0001, **=P<0.001, *=P<0.05, one-way ANOVA, Tukey post test).

FIGS. 5A-B provide the transfection efficiency for GA-pDNA, K₁₀₀-pDNA, and K₉-pDNA nanoparticles in the absence of CaCl₂ (FIG. 5A) and in the presence of 38 mmol/L CaCl₂ (FIG. 5B) at N/P ratios of 5, 10, 30, and 60 in A549 cells. RLUs refers to relative light units. Results are presented as mean ±SD (n=4) (***=P<0.0001, **=P<0.001, *=P<0.05, one-way ANOVA, Tukey post test). FIGS. 5C-D provide the transfection efficiency of GA-pDNA nanoparticles in the presence or absence of 10% fetal bovine serum [without CaCl₂ (0 mmol/L)] at N/P ratios of 5, 10, 30, and 60 in A549 cells (FIG. 5C), and in HeLa cell line [without CaCl₂ (0 mmol/L)] (FIG. 5D) without serum at N/P ratios of 5, 10, 30, and 60. RLUs refers to relative light units. Results are presented as mean ±SD (n=4) (***=P<0.0001, t test). ( [symbol] <0.0001, comparison to pDNA), one-way ANOVA, Tukey post test).

FIGS. 6A-B provide the cytotoxicity profiles of GA, K₁₀₀, K₉, and PEI in A549 cell line (FIG. 6A) and in HeLa cell line (FIG. 6B). Viability is expressed as a function of the GA, K₁₀₀, K₉, and PEI. Results are presented as mean ±SD (n=4).

FIGS. 7A-C provide stability studies (day 0, 6, and 9). FIG. 7A provides an evaluation of the particle sizes (effective diameters) of the GA-pDNA nanoparticles at day 0, day 6, and day 9 (in the absence of CaCl₂). The particle size of the nanoparticles (N/P ratio of 10) was determined by DLS in nuclease-free water (NFW). Results are presented as mean ±SD (n=3). FIG. 7B provides an evaluation of zeta potentials of the GA-pDNA nanoparticles at day 0, day 6, and day 9 (in the absence of CaCl₂). (N/P ratio of 10). Results are presented as mean ±SD (n=3). FIG. 7C provides the transfection efficiency of GA-pDNA nanoparticles at day 0, day 6, and day 9 [in the absence of CaCl₂ (0 mmol/L)] at N/P ratios of 5, 10, 30, and 60 (in A549 cell line). RLUs refers to relative light units. Results are presented as mean ±SD (n=4) (***=P<0.0001, **=P<0.001, *=P<0.05, one-way ANOVA, Tukey post test).

FIGS. 8A-B provide the results of dynamic light scattering of complexes in 4% Mannitol for GA-Poly(I:C) nanoparticles (“GA+polyI:C complexes”; FIG. 8A), and formation of GA-CpG nanoparticles (“GA+CpG complexes”) holding CpG constant and varying the GA concentration (FIG. 8B). n=4

FIGS. 9A-B provide seta potential measurements of GA complexed with Poly(I:C) at pH 7 and pH 5 (FIG. 9A), and GA complexes with CpG at pH 7 and pH 5 (FIG. 9B).

FIG. 10 provides transmission electron microscopy (TEM) of GA, Poly(I:C), GA-Poly(I:C) nanoparticles at a mass ratio of GA to Poly(I:C) of 2 (“GA+PolyI:C R2”), CpG, and GA-CpG nanoparticles at a mass ratio of GA to CpG of 5 (“GA+CpG R5”) frozen in liquid nitrogen prior to imaging

FIGS. 11A-B provide fluorescence polarization measurements for GA-Poly(I:C) nanoparticles where the GA has been labeled with Rhodamine (“Rhodamine-GA+PolyI:C”; FIG. 11A) and GA-CpG nanoparticles where the GA has been labeled with Rhodamine (“Rhodamine-GA+CpG”; FIG. 11B). Fluorescence excitation was 540 nm and emission was 620 nm. Polarization was calculated after subtracting signal produced by a standard of Poly(I:C) or CpG at the same concentration.

FIGS. 12A-B provide DNA/RNA accessibility within the GA-Poly(I:C) nanoparticles (FIG. 12A) and GA-CpG nanoparticles (FIG. 12B) as illustrated by SYBR Gold staining.

FIGS. 13A-B provide SYBR Gold fluorescence of stained GA-Poly(I:C) nanoparticles (FIG. 13A) and stained GA-CpG nanoparticles (FIG. 13B) after incubation with increasing concentrations of dextran sulfate.

FIGS. 14A-B provide the results of GA-Poly(I:C) nanoparticles (FIG. 14A) and GA-CpG nanoparticles (FIG. 14B) complexes incubated with HEK Blue hTLR3 or TLR9 respectively for 8 hours (black bars) and 20 hours (grey bars). Absorbance was read at 640 nm. Experiment was run three times with analytical duplicates or triplicates. Absorbance of sample wells were normalized to absorbance of the control (either Poly(I:C) for FIG. 14A or CpG for FIG. 14B).

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would mean “9 wt.% to 11 wt. %.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As understood by one of ordinary skill in the art, “molecular weight” (also known as “relative molar mass”) is a dimensionless quantity that can be converted to molar mass by multiplying by 1 gram/mole—for example, a polymer with a weight-average molecular weight of 5,000 has a weight-average molar mass of 5,000 g/mol.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺) ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

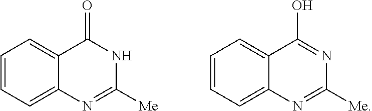 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

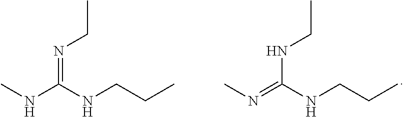

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

### The Present Technology

Delivery of polynucleotides is presently hindered by the lack of clinically translatable vectors that are both safe and efficient. Polycations (e.g., peptides, polymers, and co-polymers) that electrostatically complex with genetic material are promising vectors for gene delivery, where polylysines (“Kn”, where n is the number of lysine residues) have historically been considered effective agents for gene delivery.⁶³⁻⁶⁶

The present technology arises from the inventors surprising discovery that a glatiramoid (such as glatiramer acetate (GA) and protiramer) is a highly effective vector for delivering plasmid DNA (pDNA), polyinosine-polycytidylic acid (Poly(I:C)), and a CpG oligodeoxynucleotide. For example, as further illustrated herein, the present technology provides small, stable, positively-charged nanoparticles including GA and pDNA where such GA-pDNA nanoparticles provide excellent in vitro gene expression compared to K₉-pDNA, K₁₀₀-pDNA, and PEI-pDNA nanoparticles in A549 lung cancer cells and HeLa cervical cancer cells. Adding calcium to K₉-pDNA nanoparticles improved transfection efficiency as previously reported but unexpectedly reduced transfection efficiency of GA-pDNA nanoparticles. K₁₀₀-pDNA nanoparticles exhibited very low gene expression under all conditions tested. Furthermore, GA showed negligible cytotoxicity up to 1 mg/mL.

Thus, in an aspect, a composition is provided that includes a plurality of nanoparticles, optionally where the compostion is formulated for parenteral administration. Each nanoparticle of the plurality includes a glatiramoid as well as one or more of a polyinosine-polycytidylic acid (Poly(I:C)), a plasmid DNA (pDNA), a CpG oligodeoxynucleotide (CpG ODN), or a combination of any two or more thereof (collectively hereafter referred to as “polynucleotide”). The plurality of nanoparticles of any embodiment herein may have an intensity-weighted average diameter as determined by dynamic light scattering of about 20 nm, about 40 nm, about 60 nm, about 80 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500, or any range including and/or in between any two of these values. For the sake of clarity, the composition may or may not include other types of nanoparticles than the nanoparticles of the plurality (e.g., other pluralities of nanoparticles that are not those nanoparticles that include a glatiramoid and a polynucleotide).

A glatiramoid is a synthetic heterogenous polypeptide mixture that includes four natural amino acids, L-glutamic acid, L-alanine, L-lysine, and L-tyrosine, in a distinct molar ratio of 0.14:0.43:0.09:0.34, respectively. Examples of a glatiramoid include, but are not limited to, glatiramer acetate (GA) and protirmamer. In any embodiment herein, the glatiramoid of the composition may have a weight average molecular weight of about 5,000 to about 18,000; thus, the glatiramoid may have a weight average molecular weight of about 5,000, about 6,000, about 7,000, about 8,000, about 9,000, about 10,000, about 11,000, about 12,000, about 13,000, about 14,000, about 15,000, about 16,000, about 17,000, about 18,000, or any range including and/or in between any two of these values. Thus, by way of example, the glatiramoid of any embodiment herein may include glatiramer acetate and possess a weight average molecular weight of about 5,000 to about 9,000.

In any embodiment disclosed herein, the plurality of nanoparticles may be configured to possess a ratio of cationic charges of the glatiramoid (N) to phosphate anionic charges of polynucleotide (P) of about 0.5:1 to about 100:1 (referred to herein also as “an N/P ratio”). Thus, the plurality of nanoparticles may be configured to possess an N/P ratio of about 0.5:1, about 1:1, about 2:1, about 3:1, about 4:1, about 5:1, about 6:1; about 7:1, about 8:1, about 9:1, about 10:1, about 11:1, about 12:1, about 13:1, about 14:1, about 15:1, about 16:1, about 17:1, about 18:1, about 19:1, about 20:1, about 25:1, about 30:1, about 40:1, about 50:1, about 60:1, about 70:1, about 80:1, about 90:1, about 100:1, or any range including and/or inbetween any two of these values.

In any embodiment disclosed herein, the plurality of nanoparticles may be configured to possess a mass ratio of glatiramoid to polynucleotide of about 0.5:1 to about 30:1; thus, the plurality of nanoparticles may be configured to possess a mass ratio of glatiramoid to polynucleotide of about 0.5:1, about 1:1, about 2:1, about 3:1, about 4:1, about 5:1, about 6:1; about 7:1, about 8:1, about 9:1, about 10:1, about 11:1, about 12:1, about 13:1, about 14:1, about 15:1, about 16:1, about 17:1, about 18:1, about 19:1, about 20:1, about 25:1, about 30:1, or any range including and/or inbetween any two of these values.

The Poly(I:C) of any embodiment herein may have a weight average number of base pairs of about 0.2 kb to about 8 kb. In general, a “low molecular weight Poly(I:C)” (or “LMW Poly(I:C)”) typically has a weight average number of base pairs of about 0.2 kb to about 1 kb, and in general a “high molecular weight Poly(I:C)” (or “HMW Poly(I:C)”) typically has a weight average number of base pairs of about 1.5 kb to about 8 kb. Thus, the Poly(I:C) of any embodiment disclosed herein may have a weight average number of base pairs of about 0.2 kb, about 0.3 kb, about 0.4 kb, about 0.5 kb, about 0.6 kb, about 0.7 kb, about 0.8 kb, about 0.9 kb, about 1.0 kb, about 1.1 kb, about 1.2 kb, about 1.3 kb, about 1.4 kb, about 1.5 kb, about 1.6 kb, about 1.7 kb, about 1.8 kb, about 1.9 kb, about 2.0 kb, about 2.2 kb, about 2.4 kb, about 2.6 kb, about 2.8 kb, about 3.0 kb, about 3.5 kb, about 4.0 kb, about 4.5 kb, about 5.0 kb, about 5.5 kb, about 6.0 kb, about 6.5 kb, about 7.0 kb, about 7.5 kb, about 8.0 kb, or any range including and/or in between any two of these values.

The CpG ODN of any embodiment herein may include a Class A CpG ODN, a Class B CpG ODN, a Class C CpG ODN, or a combination of any two or more thereof. The CpG ODN of any embodiment herein may include Class B CpG ODN 1825, Class B CpG ODN 2006, Class B CpG ODN BW006, Class B CpG ODN 1668, Class A CpG ODN 1585, Class A CpG ODN 2216, Class A CpG ODN 2336, Class C CpG ODN 2395, Class C CpG ODN M362, or a combination of any two or more thereof.

The pDNA of any embodiment herein may include angiotensin II type 2 receptor pDNA (pAT2R), pDNA encoding anti-HER2 antibody, pDNA encoding murine interferon a (mIFN-α), or a combination of any two or more thereof.

The composition of any embodiment herein may be at a pH of about 5 to about 10. Thus, the composition may be at a pH of about 5.0, about 5.5, about 6.0, about 6.5, about 7.0, about 7.5, about 8.0, about 8.5, about 9.0, about 9.5, about 10, or any range including and/or in between any two of these values. The composition of any embodiment herein may include a concentration of CaCl₂ that is no greater than about 1 mM. The composition of any embodiment herein may include a concentration of CaCl₂ that is no greater than about 1 nanomolar. The composition of any embodiment herein may include a concentration of CaCl₂ that is about 0 nanomolar.

The composition of any one of the herein-described embodiments may include an effective amount of the plurality of nanoparticles. Thus, the composition may be a pharmaceutical composition. “Effective amount” refers to the amount of the nanoparticles required to produce a desired effect in a subject. One example of an effective amount includes amounts or dosages that yield acceptable toxicity and bioavailability levels for therapeutic (pharmaceutical) use. Further, the pharmaceutical composition may be packaged in unit dosage form. Generally, a unit dosage will vary depending on patient considerations. Such considerations include, for example, age, protocol, condition, sex, extent of disease, contraindications, concomitant therapies and the like. As used herein, a “subject” or “patient” is a mammal, such as a cat, dog, rodent or primate. Typically the subject is a human, and, preferably, a human suffering from or suspected of suffering from pain. The term “subject” and “patient” can be used interchangeably. In a further related aspect, a method is provided that includes administering an effective amount of a composition any embodiment disclosed herein to a subject, where the administering step includes parenteral administration of the composition to the subject. Such a method may be used to deliver a gene to a subject.

Parenteral or systemic administration includes, but is not limited to, subcutaneous, intravenous, intraperitoneal, and intramuscular, injections. The compositions, pharmaceutical compositions, and medicaments of the present technology formulated for parenteral administration may be prepared by mixing one or more components with pharmaceutically acceptable carriers, excipients, binders, diluents, or the like (collectively, referred to herein as “a pharmaceutically acceptable carrier”). The following dosage forms are given by way of example and should not be construed as limiting the instant present technology.

Pharmaceutical formulations and medicaments of the present technology may be prepared as liquid suspensions or solutions using a sterile liquid, such as, but not limited to, an oil, water, an alcohol, and combinations of any two or more of these. Pharmaceutically suitable surfactants, suspending agents, emulsifying agents, may be added for parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until, for example, a desired outcome is observed. The compounds of the present technology may be administered to a patient at dosage levels in the range of about 0.001 to about 1,000 mg per day. For a normal human adult having a body weight of about 70 kg, a dosage in the range of about 0.001 to about 100 mg per kg of body weight per day may be sufficient. The specific dosage used, however, can vary or may be adjusted as considered appropriate by those of ordinary skill in the art. For example, the dosage can depend on a number of factors including the requirements of the patient, the severity of the pain and the pharmacological activity of the compound being used. The determination of optimum dosages for a particular patient is well known to those skilled in the art. Various assays and model systems can be readily employed to determine the therapeutic effectiveness of the treatment according to the present technology.

The compositions of the present technology may also be administered to a patient along with other conventional therapeutic agents that may be useful in treatment. The administration of the one or more other conventional therapeutic agents(s) may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective.

The examples herein are provided to illustrate advantages of the present technology and to further assist a person of ordinary skill in the art with preparing or using the compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

### Materials

Plasmid DNA (pDNA) encoding firefly luciferase (pGL3, 4818 bp) was purchased from Promega (Madison, Wis.). The pDNA purity level was determined by UV-Spectroscopy and agarose gel electrophoresis. Glatiramer Acetate [Teva Pharmaceuticals USA Inc (Copaxone®)] 20 mg per mL samples (Dosage form: injection, solution) were donated by Sharon G. Lynch, M.D. Department of Neurology, KUMC. K₉ peptide (KKKKKKKKK (SEQ ID NO: 1); Mw=1170.65 Da), the C-terminal of the peptide is synthesized as an amide was obtained from Biomatik Corporation (Cambridge, Ontario, Canada) (Purity>95%). K₁₀₀ [(KKKKKKKKKK)ₙ₌₁₀ (SEQ ID NO: 2), Mw =16,000 Da] was obtained from Alamanda Polymers. Inc., Huntsville, Ala., USA). Branched polyethyleneimine (PEI, 25 kDa) was purchased from Sigma-Aldrich (Milwaukee, Wis.). CpG ODN 1826 and LMW Poly(I:C) were purchased from Invivogen (San Diego, Calif.). A549 cell line (carcinogenic human alveolar basal epithelial) was purchased from American Type Culture Collection (ATCC; Rockville, Md.). HeLa cell line (cervical cancer cells) was a gift from Tamura Lab (Masaaki Tamura, Ph.D., Kansas State University, College of Veterinary Medicine [obtained from American Type Culture Collection (ATCC; Rockville, Md.)]. F-12K Nutrient Mixture, Kaighn's modified with L-glutamine was purchased through Cellgro (Mediatech, Inc., Manassas, Va.). Dulbecco's Modified Eagle's Medium (DMEM) was obtained from Invitrogen/Life Technologies (Gibco®) (Grand Island, N.Y. 14072, USA). Fetal bovine serum (FBS) was purchased from Hyclone (Logan, Utah). Penicillin/Streptomycin was obtained from MB Biomedical, LLC (Solon, Ohio). Trypsin-EDTA was purchased from Invitrogen (Carlsbad, Calif.). Luciferase Assay System Freezer Pack and CellTiter 96® AQueous one solution cell proliferation assay (MTS) were purchased from Promega (Madison, Wis.). BCA Protein Assay Reagent (bicinchoninic acid) was purchased from Thermo Fisher Scientific Inc. Tris-acetate-EDTA (TAE) Buffer (10×) was purchased from Promega (Madison, Wis.). Sterile water (DNase, RNase-free) was purchased from Fisher Scientific. Calcium chloride dihydrate (CaCl₂. 2H₂O) was purchased from Fisher Scientific. Agarose (Medium-EEO/Protein Electrophoresis Grade) was obtained from Fisher Scientific. Bench Top DNA Ladder was obtained from Promega (Madison, Wis.). SYBR Green I Nucleic Acid Gel Stain was obtained from Invitrogen (Carlsbad, Calif.). Agarose (Medium-EEO/Protein Electrophoresis Grade) was purchased from Fisher Scientific. Other organic chemicals used for this work were obtained from Fisher Scientific.

### Methods

Rhodamine labeled GA. GA [Teva Pharmaceuticals USA Inc (Copaxone®)] dialyzed in water was reacted with 2 equivalents of Rhodamine B N-hydroxysuccinimide (NETS) ester (7000 was used as the molecular weight of GA for calculation) in CPB buffer (10 mM citrate, 20 mM phosphate, 40 mM borate) pH 7.5 with 20% dimethyl sulfoxide. The reaction was carried out at room temperature for 4 hours protected from light with gentle agitation. To separate labeled drug from excess dye, the reaction mixture was placed into dialysis cassettes with 2 kDa MWCO and dialyzed against 5% dimethylformamide in water at pH 2, followed by 0.5 M LiCl solution, and finally water. Dialysis was performed sequentially in each buffer for 24 hours with one buffer change in between for total of 72 hours. The resulting reaction solution was characterized by HPLC and lyophilized. The number of dye labeled onto GA was determined by constructing a calibration curve based on the fluorescence of Rhodamine B NHS ester at various concentrations and comparing the fluorescence of the labeled product to the calibration curve. The fluorescence experiments were performed using Synergy™ H4 Microplate Reader (BioTek, Winooski, Vt.) with 540/25 nm excitation filter and 620/40 nm emission filter.

Nanoparticle Formation. GA-pDNA nanoparticles, K₁₀₀-pDNA nanoparticles, and K₉-pDNA nanoparticles were prepared by adding 15 μL of the GA, K₁₀₀, or K₉ solutions at polymer nitrogen to pDNA phosphate (N/P) ratios of 1, 5, 10, 20, 30, and 60 to 10 μL (0.1 μg/μL) of pDNA (TAE Buffer (1×) was used as a solution for DNA storage), followed by repeated pipetting for 20-25 seconds. At that point, 15 μL of CaCl₂ (0 or 38 mmol/L) was added to determine the effect of calcium. After formulating, samples were stored at 4° C. for ˜20 minutes. PEI-pDNA nanoparticles were prepared by adding 15 μL of PEI solution (N/P ratio of 10) to 10 μL (0.1 μg/μL) of pDNA followed by pipetting for 20-25 seconds. After preparing PEI-pDNA nanoparticles, they were stored at 4° C. for ˜20 minutes. All nanoparticles in this study were prepared immediately before each experiment.

Nanoparticles (also herein referred to as “complexes”) including GA with CpG ODN 1826 (“GA-CpG complexes”) or LMW Poly(I:C) (“GA-Poly(I:C) complexes”) were prepared by adding equal volumes of pre-diluted GA and pre-diluted CpG or PolyI:C followed by repeated pipetting for 30 seconds. The complexes were then stored at room temperature for a minimum of 20 minutes before measurements or cell culture use. Complexes were prepared at varying mass ratios of 1, 2, 3, 4, 5, 10, 20 that represent mass of GA divided by the complex partner, CpG or Poly(I:C), holding the CpG or Poly(I:C) concentration constant while varying the GA concentration. Mass ratio was utilized rather than a N:P ratio due to heterogeneity of the components. Similar procedures were used for generating Rhodamine-labeled GA-CpG complexes and Rhodamine-labeled GA-Poly(I:C).

Agarose Gel Electrophoresis. GA-pDNA nanoparticles, K₁₀₀-pDNA nanoparticles, and K₉-pDNA nanoparticles were prepared as defined above and subsequently, 4 μL of Tris-acetate-EDTA (TAE) buffer was added. Then, 4 μL of SYBR Green 1 was mixed with the nanoparticles. Afterward, the mixture was stored at 4° C. for 20-25 minutes. Then, 7μL of 6X DNA loading dye (Takara Bio Inc., Japan) was added. A one kb DNA ladder (Promega, Madison, Wis.) was used. The mixture solutions were loaded onto a 1% agarose gel, and electrophoresed for 30 minutes at 110 V.

For GA-CpG and GA-Poly(I:C) complexes 4 μL 6× DNA loading dye (Takara Bio Inc., Japan) was added to 10 μL it of the complex and subsequently 12 μL it was loaded onto a 3% agarose gel, and electrophoresed for 25 minutes at 100 V. CpG and PolyI:C alone were run as controls and a 1 kb bench top DNA ladder (Promega, Madison, Wis.) was used. The gel was stained using SYBR Gold (Invitrogen, Carlsbad, Calif.) in TAE buffer for 25 minutes, shaking at room temperature then imaged on Alphalmager (Protein Simple, San Jose, Calif.).

Particle Size and Zeta Potential. The particle size [effective diameter (nm)] of GA-pDNA nanoparticles, K₁₀₀-pDNA nanoparticles, and K₉-pDNA nanoparticles in the presence and absence of CaCl₂ was determined by dynamic light scattering (DLS, Brookhaven Instruments, Holtsville, N.Y.). The zeta potentials of the nanoparticles were measured by Zeta PALS dynamic light scattering (Brookhaven Instrument, Holtsville, N.Y.). All samples intended for particle size measurements were prepared using Phosphate Buffered Saline (PBS), Serum-Free Media (SFM) and Nuclease-Free Water (NFW). All samples intended for zeta potential measurements were prepared using KCl (1 mM).

The effective radius (nm) of GA-CpG or GA-Poly(I:C) complexes was determined by dynamic light scattering (DynaPro, Wyatt Technology, Santa Barbara, Calif.). Samples for particle sizing were prepared in 4% mannitol (Sigma Aldrich, St. Louis, Mo.). Measurements were conducted after a minimum of 20 minutes of incubation at room temperature. The zeta potentials were measured by Zeta PALS dynamic light scattering (Brookhaven Instrument, Holtsville, N.Y.) where GA-CpG or GA-Poly(I:C) samples for zeta potential measurements were prepared in 4% mannitol and diluted into 1 mM KCl for analysis.

Fluorescence Polarization. Fluorescence polarization measurements were taken on Synergy H4 microplate reader (BioTek, Winooski, Vt.).

For studies involving Rhodamine-labeled GA-CpG complexes and Rhodamine-labeled GA-Poly(I:C) complexes, first a standard curve of Rhodamine-labeled GA and standards containing identical concentration of CpG or Poly(I:C) without Rhodamine-labeled GA for each complex were prepared. Then, 200 μL of the Rhodamine-labeled GA-CpG complexes, Rhodamine-labeled GA-Poly(I:C) complexes, Rhodamine-labeled GA, CpG, or Poly(I:C) were added to a 96 well, black microplate (Corning, Corning, N.Y.). Using fluorescence polarization settings on the plate reader, the excitation filter was set to 485 nm/20 nm and emission filter to 620 nm/40 nm. To calculate the polarization, first the parallel and perpendicular values for the standards (CpG or Poly(I:C) alone) are subtracted from their respective complexes (Rhodamine-labeled GA-CpG complexes or Rhodamine-labeled GA-Poly(I:C) complexes), then polarization subtracting any background from CpG or Poly(I:C) alone was calculated using the following equation (Eq. 1):

$$P = \frac{I \vert \vert - I ⊥}{I \vert \vert + I ⊥} \qquad Eq . 1$$

Transmission Electron Microscopy (TEM). TEM images were captured using FEI Tecnai F20 XT Field Emission Transmission Electron Microscope at the University of Kansas Microscopy and Analytical Imaging Laboratory. Complexes or individual components were added to carbon coated grids and touched on a Kimwipe to remove excess liquid, then immediately dipped into liquid nitrogen prior to imaging

The Effect of Dextran Sulfate on the Stability of the Nanoparticles. The degree of pDNA accessibility following complexation with GA, K₁₀₀, K₉, or PEI was assessed using the double-stranded-DNA-binding reagent SYBR Green (Invitrogen). Briefly, 10 μL (0.1 mg/mL) of pDNA was mixed with 15 μL of GA, K₁₀₀, K₉, or PEI solution, then 75 μL of deionized water solution was added. The samples were left for 30 minutes at room temperature before use. After incubation, 20 μL of 10× SYBR Green solution was added. The samples were incubated for 10 minutes. After the incubation, dextran sulfate solution (120 μL) of stock concentration of 0, 0.01, 0.1, and 1 mg/mL was added to the nanoparticle suspensions to yield final concentrations of 0, 5, 50, and 500 μg/μL, then incubated for 30 minutes at room temperature. Next, 100 μL of each sample was added to one well of a 96-well cell culture plate. The fluorescence was measured using a fluorescence plate reader (SpectraMax M5; Ex., 250 nm; Em, 520 nm).

Similarly, GA-CpG and GA-Poly(I:C) complexes were made as described above and, after a minimum of 20 minutes, 135 μL of complex sample was added to a 96-well plate in triplicate then 15 μL of 10× SYBR gold was added and mixed well. After ˜5 minutes the fluorescence was measured using Synergy H4 microplate reader (Ex. 495 nm, Em. 537 nm) (BioTek, Winooski, Vt.). For studying the effect of dextran sulfate, 90 μL of pre-formed GA-CpG or GA-Poly(I:C) complexes were added to a 96-well plate followed by 10 μL of dextran sulfate in various concentrations and mixed well. After 20-30 minutes of RT incubation, 11 of 10× SYBR Gold was added and 5 minutes later the plate was read as described previously.

### Cell Culture.

A549 and HeLa cells: A549 lung cancer and HeLa cervical cancer cell lines were grown in F-12K Nutrient Mixture media (Kaighn's modified with L-glutamine, for A549) and Dulbecco's Modified Eagle's Medium (DMEM, for HeLa) with 1% (v/v) Penicillin/Streptomycin and 10% (v/v) fetal bovine serum (FBS) at 37° C. in 5% CO₂ humidified air.

Jaws II cells: Jaws II cells (ATCC Manassas, Va.) were cultured in RPMI, 10% FBS (Atlanta Biologicals), 1% penicillin-streptomycin (P/S, MP Biomedicals), and 5 ng/mL GM-CSF (Tonbo Biosciences). Jaws II cells were plated at 2.5×10⁵ cells/well, at 270 μL/well in a 96 well plate and allowed to adhere for an hour before adding treatments. Then, 30 μL of 10× complex or individual component was added to each well. Additionally to assess cell stability in the presence of various buffers, 30 μL of either 4% mannitol, 5% glucose, NFW, or saline was added into 270 μL media+cells and images were taken on an inverted microscope (Accu-Scope, Hicksville, N.Y.) as well as resazurin assay to assess cell metabolism.

Bone Marrow Derived Dendritic Cells: Five-week-old C57BL/6J mice were purchased from Jackson Laboratories and housed under specified, pathogen-free conditions at The University of Kansas. All protocols involving mice were approved by the Institutional Animal Care and Use Committee at The University of Kansas. Mice were sacrificed and their femurs were collected. The ends of the femur were clipped, and the bone marrow was flushed out using a 21-gauge needle attached to a 5 mL syringe containing RPMI supplemented with 1% penicillin-streptomycin. Cells were collected and centrifuged for 7 minutes at 1,350 rpm at 4° C. The supernatant was removed, replaced with red cell lysis buffer, and incubated at room temperature for 10 minutes. Lysis was stopped with 6× volume of cold complete medium (RPMI, 10% FBS, 1% penicillin-streptomycin). The cell solution was passed through a 70 μm nylon cell strainer and centrifuged for 5 minutes at 1,700 rpm and 4° C. The supernatant was removed and replaced with complete medium, and cells were plated at approximately 2×10⁶ cells per T-75 culture flask in 12 mL complete medium spiked with 20 ng/mL GM-CSF. On day 3, the medium was removed to discard any floating cells, and 12 mL of media with fresh GM-CSF was added to the cells. On day 8, the media with cells were collected and the bottom of the flask was thoroughly rinsed to collect any loosely adherent cells. BMDCs were then plated at 2.5×10⁵ cells/well and treated as previously described for the Jaws II culture conditions.

HEK Blue cells: HEK-Blue TLR9, TLR3, and Null cell lines (Invivogen, Calif.) were grown in Dulbecco's Modified Easle's Medium (DMEM; Corning, N.Y.) supplemented with 10% FBS, 1% penicillin-streptomycin, and the selective antibiotics according to the manufacturer's protocol. HEK-Blue TLR cells allow for the study of TLR activation by observing the stimulation of SEAP, a protein associated with downstream activation of TLRs. At 50-80% confluency, cells were harvested and resuspended in HEK detection media (Invivogen, Calif.) and 180 uL was seeded into 96-well plates at ˜8×10̂5 cells/well. 20 uL of treatment were added to respective wells and the plate was incubated at 37° C., 5% CO₂ for at least 6 hours or until color change. Absorbance readings were measured at 640 nm. Null cells were used as the control.

Metabolism. Cell viability was inferred from metabolic activity measured by the resazurin assay. Wells were washed to remove as much of the treatments as possible and 100 μL of RPMI and 20 μL of 0.01% resazurin were added to the wells. Plates were incubated at 37° C. for one or two hours, and the fluorescence was measured at ex/em 560/590 nm using a Synergy H4 microplate reader (BioTek, Winooski, Vt.). Data within each stimulation group was normalized to the untreated media control at their respective time points.

Transfection Study. A549 and HeLa cell lines were cultured in 96-well plates for 24 hours prior to transfection. The concentration of the cells in every well was approximately 1,000,000 cells/mL. The wells were washed once with serum-free media (SFM), and later a 100 sample (which consisted of 20 μL of GA-pDNA nanoparticles, K₁₀₀-pDNA nanoparticles, or K₉-pDNA nanoparticles and 80 μL of SFM) was added to each well. Then, a 96-well plate was incubated for 5 hours in an incubator. After the incubation, the sample was replaced with 100 μL of fresh serum medium and then incubated again for approximately 48 hours. To determine the gene expression of the nanoparticles, the Luciferase Reporter Assay from Promega was used. The results of the transfections were expressed as Relative Light Units (RLU) per milligram (mg) of cellular protein, and PEI-pDNA was used as a control. BCA Protein Assay Reagent (bicinchoninic acid) was used to measure total cellular protein concentration in the cell extracts. The Luciferase Assay and BCA were measured by a microplate reader (SpectraMax; Molecular Devices Crope, Calif.).

TNF-α ELISA. TNF-α expression by dendritic cells was measured by ELISA (R&D systems, Minneapolis, Minn.) per manufacturer instructions.

Cytotoxicity Assay. Cytotoxicity of GA, K₁₀₀, and K₉, PEI, and CaCl₂ was determined using a CellTiter 96® AQueous Non-Radioactive Cell Proliferation Assay (MTS) obtained from Promega (Madison, Wis.). A549 and HeLa cells were cultured in a 96-well plate as described previously. Cells were treated with the samples for ˜24 hours. Then, the media were replaced with a mixture of 100 μL of fresh serum medium and 20 μL of MTS. The plate was incubated for 3-4 hours in the incubator. To determine cell viability, the absorbance of each well was measured by a microplate reader (SpectraMax; Molecular Devices Crope, CA) at 490 nm and normalized to untreated control cells.

Stability Study. GA-pDNA nanoparticles were incubated for 0, 3, and 6 days in the refrigerator (4° C.). The particle size, zeta potential, and transfection efficiency were measured for GA-pDNA nanoparticles at 0, 2, and 3 days.

Statistical Analysis. Data were analyzed using GraphPad software. A statistical evaluation comparing the significance of the difference in gene expression (RLUs/mg protein) between the means of two data sets was performed using a t-test. One-way ANOVA with Tukey post-test was used to analyze the differences when more than two data sets were compared. The Pearsons correlation coefficient was calculated with one-tailed probability.

### Results

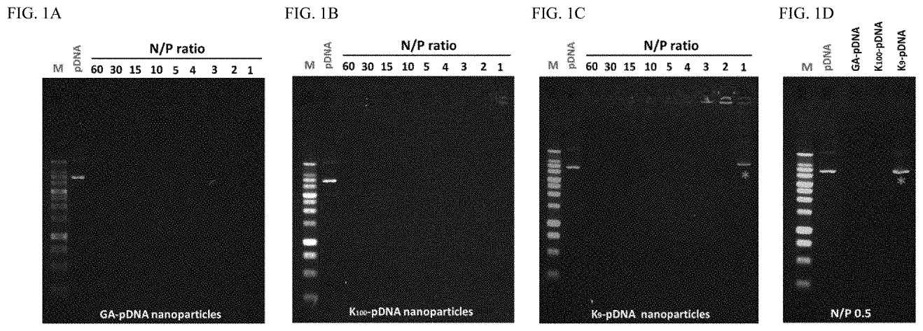

Agarose gel electrophoresis indicated GA could immobilize pDNA. The formation of GA-pDNA and K₁₀₀-pDNA nanoparticles was observed via agarose gel electrophoresis. The GA-pDNA, K₁₀₀-pDNA, K₉-pDNA, PEI-pDNA nanoparticles were prepared by mixing pDNA (pGL3) with each polycation at different N/P ratios (1, 2, 3, 4, 5, 10, 15, 30, and 60; see FIG. 1). Naked pDNA was used as a control. Agarose gel electrophoresis studies showed that GA-pDNA nanoparticles (FIG. 1A), K₁₀₀-pDNA nanoparticles (FIG. 1B), and K₉-pDNA nanoparticles (FIG. 1C) were robust enough to immobilize pDNA completely across the range of N/P ratios (1 to 60). However, K₉ did not completely complex pDNA at an N/P ratio of 1. FIG. 1D illustrates a lower N/P ratio of 0.5, where GA and K₁₀₀ were still able to immobilize pDNA completely, and K₉ did not. Our data suggested that GA co-polymer and K₁₀₀ CPP were able to complex pDNA and form stable nanoparticles at very low concentrations. Nevertheless, K₉ CPP was insufficient to complex pDNA at these concentrations.

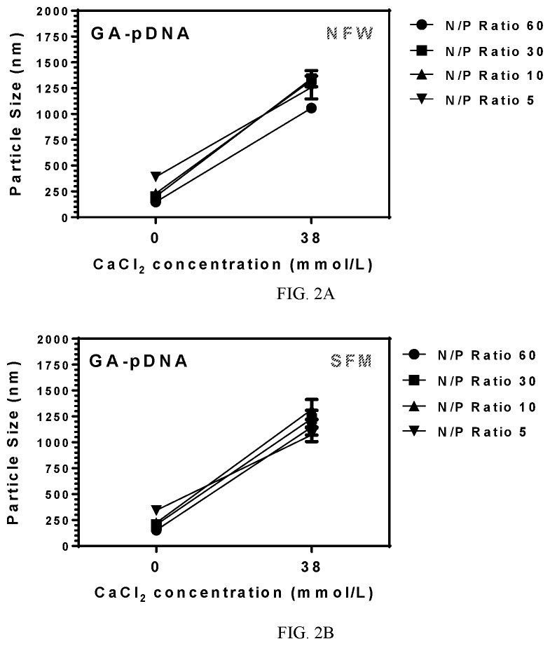

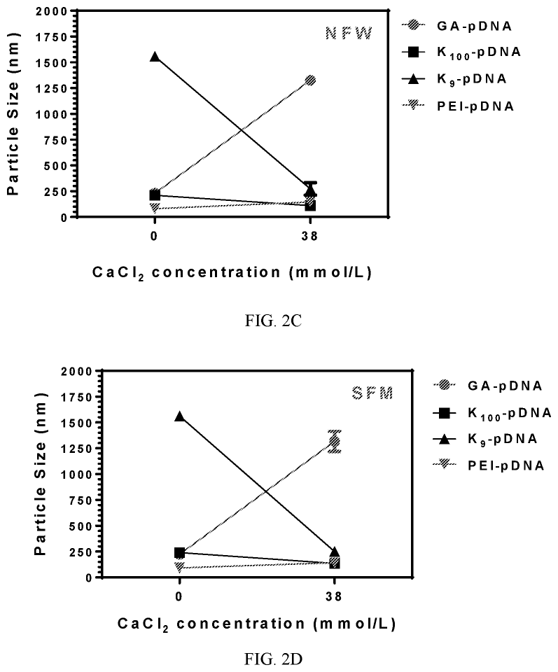

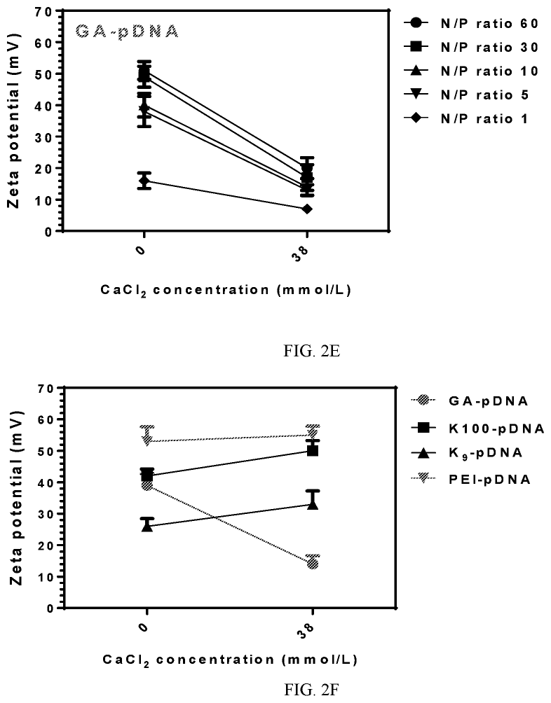

GA is capable of packaging pDNA into small, cationic nanoparticles. The effect of CaCl₂ on the size of GA-pDNA nanoparticles was determined at N/P ratios of 5, 10, 30, and 60 in nuclease-free water (NFW) (FIG. 2A), and in serum-free F-12 media (SFM) (FIG. 2B). GA-pDNA nanoparticles showed an increase in particle size with the addition of CaCl₂ in both NFW and SFM. This figure shows the size of the GA-pDNA, K₁₀₀-pDNA, and K₉-pDNA nanoparticles as well as PEI-pDNA nanoparticles with or without CaCl₂ in NFW (FIG. 2C) or in SFM (FIG. 2D). In the presence of calcium, the GA-pDNA and PEI-pDNA nanoparticles showed an increase in size (from ˜200 to 1300 nm and from ˜100 to 140 nm respectively). Conversely, K₁₀₀-pDNA nanoparticles showed a slight decrease in the particle size in the presence of calcium (from ˜200 to 100 nm). On the other hand, K₉-pDNA nanoparticles in the presence of calcium showed a substantial decrease in the particle size (from ˜1500 to 250 nm).

GA-pDNA nanoparticles exhibited a positive charge. FIG. 2E indicates the charge of GA-pDNA nanoparticles (N/P ratio of 1 to 60) generally decreased in the presence of calcium (from ˜40 to 15 mV for N/P ratios 5 to 60; and from ˜16 to 7 mV for N/P ratio 1). The net positive charge of the nanoparticles confirms the GA, being in excess, forms the shell of the nanoparticles. FIG. 2F illustrates a comparision of the zeta potential of the different nanoparticle types formulated using an N/P ratio of 10. Interestingly, K₁₀₀-pDNA, K₉-pDNA, and PEI-pDNA nanoparticles showed an increase in the zeta potential in the presence of calcium (from 42 to 50 mV, from 26 to 33 mV, from ˜49 to 55 mV respectively). Conversely, the GA-pDNA nanoparticles showed a decrease in the zeta potential value when calcium was included.

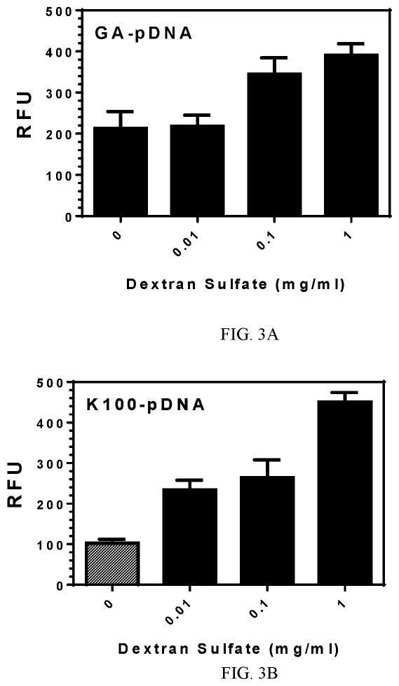

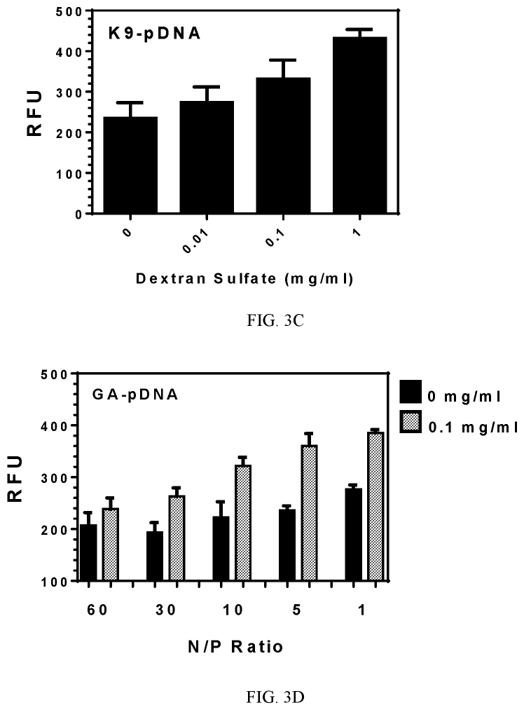

GA-pDNA nanoparticles package DNA in a manner similarly to K₉. The SYBR Green test provides an easy, high-throughput, non-destructive method for examining pDNA accessibility within polyelectrolyte complex nanoparticles. FIGS. 3A-D show the fluorescence of the GA-pDNA nanoparticles (FIG. 3A), K₁₀₀-pDNA nanoparticles (FIG. 3B) and K₉-pDNA nanoparticles (FIG. 3C) when challenged with different dextran sulfate concentrations (0, 0.01, 0.1, and 1 mg/mL). The rise in relative fluorescence units (RFU) indicated increased accessibility of pDNA as the dextran sulfate concentration increased. The RFU of the GA-pDNA, K₁₀₀-pDNA, and K₉-pDNA nanoparticles increased from ˜200 to ˜400 RFU, ˜100 to ˜450 RFU, and ˜200 to ˜450 RFU respectively as the dextran sulfate increased from 0 to 1 mg/ml. FIG. 3D displays the SYBR Green fluorescence of the GA-pDNA nanoparticles (at N/P ratios of 1, 5, 10, 30, and 60) in the presence and absence of 0.1 mg/mL of dextran sulfate.

GA-pDNA nanoparticles potently transfect cells. The gene expression mediated by GA-pDNA, polylysine-pDNA, and PEI-pDNA nanoparticles in A549 cells was studied as a function of N/P ratio (5, 10, 30, and 60). The in vitro transfection efficiency of the nanoparticles was studied using two different human cancer cell lines including A549 and HeLa. Luciferase gene expression was evaluated 48 hours after the transfection.

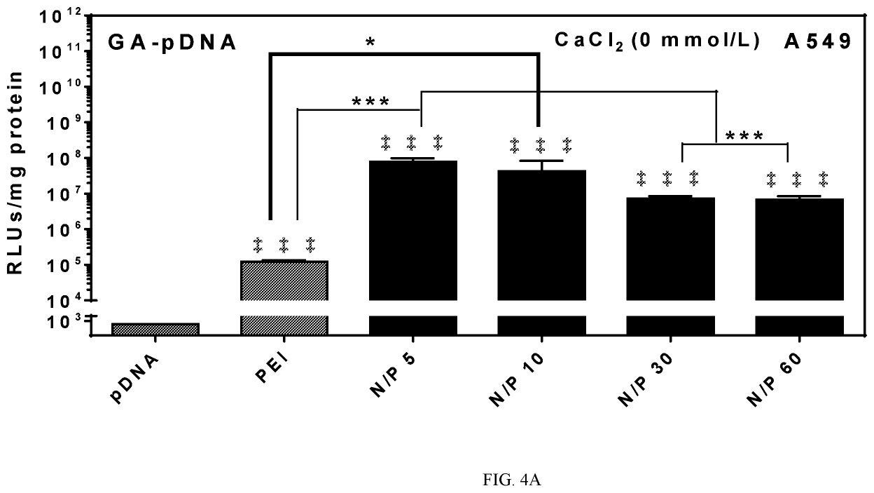

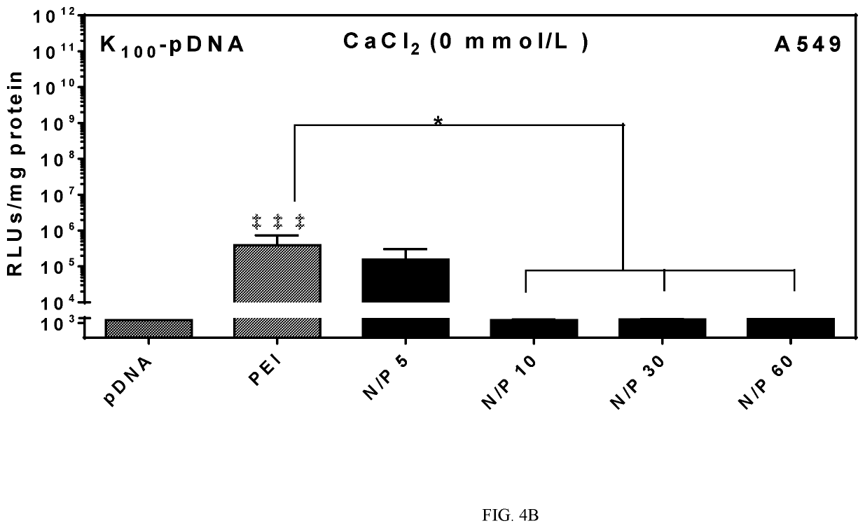

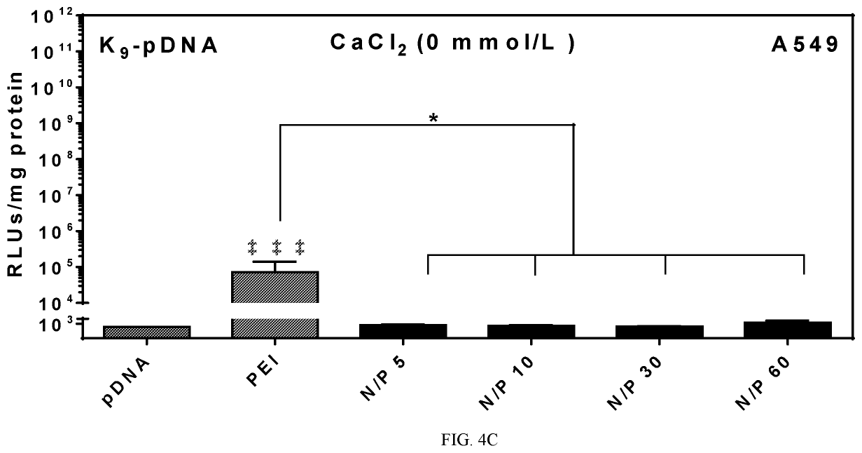

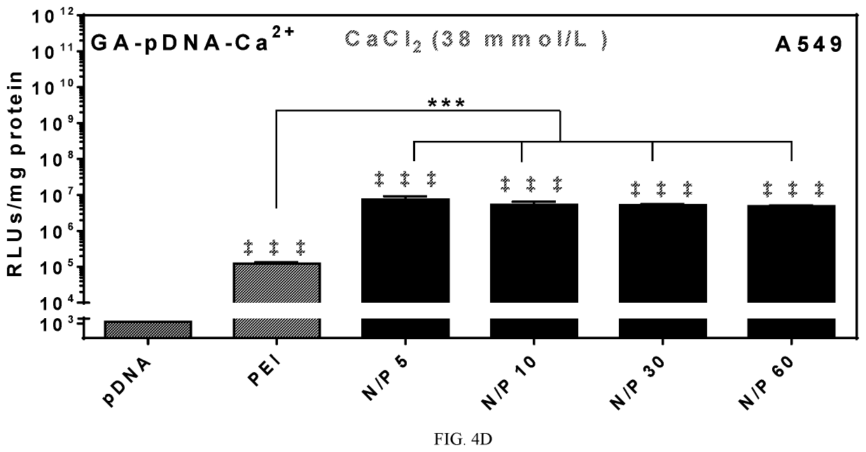

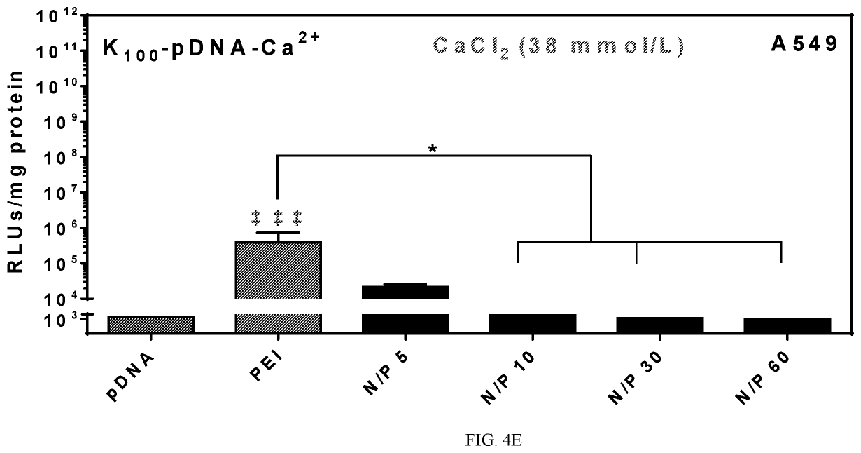

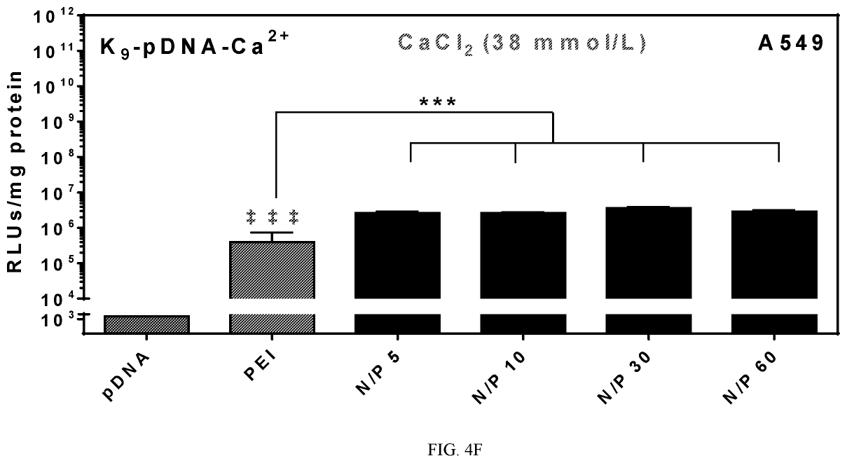

FIGS. 4A-C show the transfection efficiency of GA-pDNA (FIG. 4A), K₁₀₀-pDNA (FIG. 4B), and K₉-pDNA nanoparticles (FIG. 4C) in the absence of calcium at N/P ratios of 5, 10, 30, and 60 in A549 cells. PEI-pDNA nanoparticles (N/P ratio 10) were used as a positive control. Gene expression of the GA-pDNA nanoparticles was significantly higher than PEI-pDNA nanoparticles and the free pDNA. The gene expression of the K₁₀₀-pDNA and K₉-pDNA nanoparticles was significantly lower than PEI-pDNA nanoparticles. FIGS. 4D-F display the transfection efficiency of the GA-pDNA (FIG. 4D), K₁₀₀-pDNA (FIG. 4E), and K₉-pDNA nanoparticles (FIG. 4F) in the presence of calcium at N/P ratios of 5, 10, 30, and 60 in A549 cells. Here, the gene expression of GA-pDNA and K₉-pDNA nanoparticles was significantly higher than PEI-pDNA nanoparticles and free pDNA. Again, the transfection efficiency of the K₁₀₀-pDNA nanoparticles were significantly lower than PEI-pDNA nanoparticles.

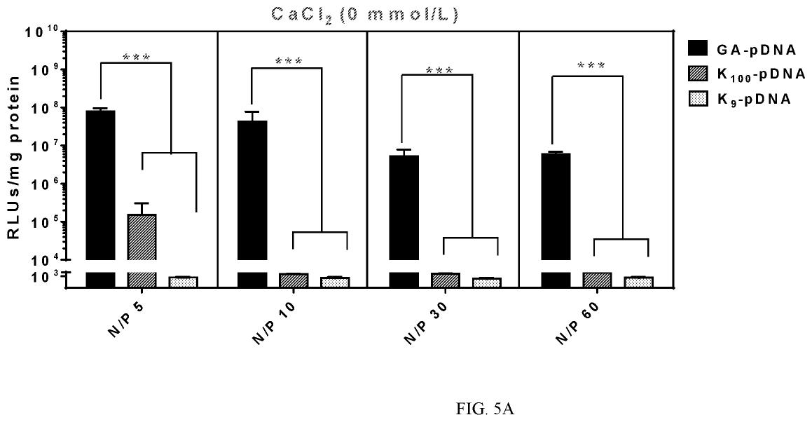

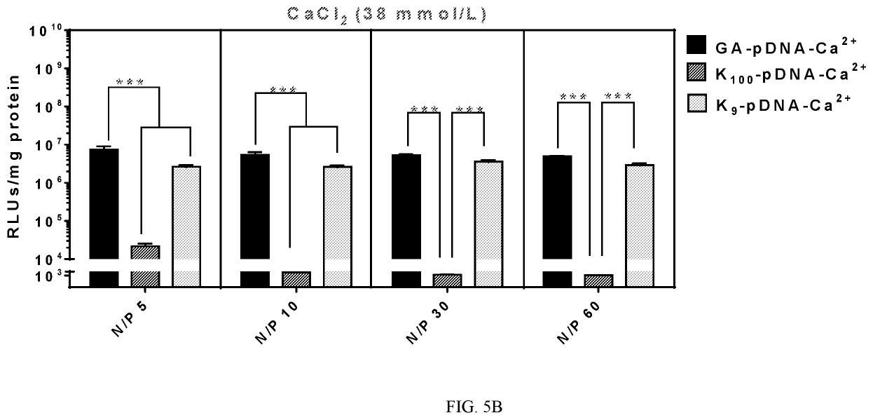

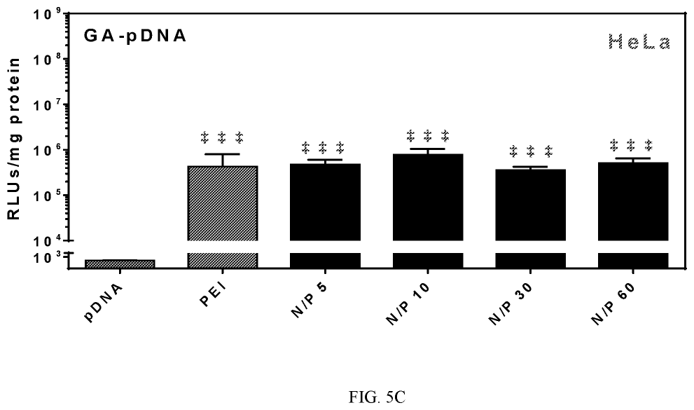

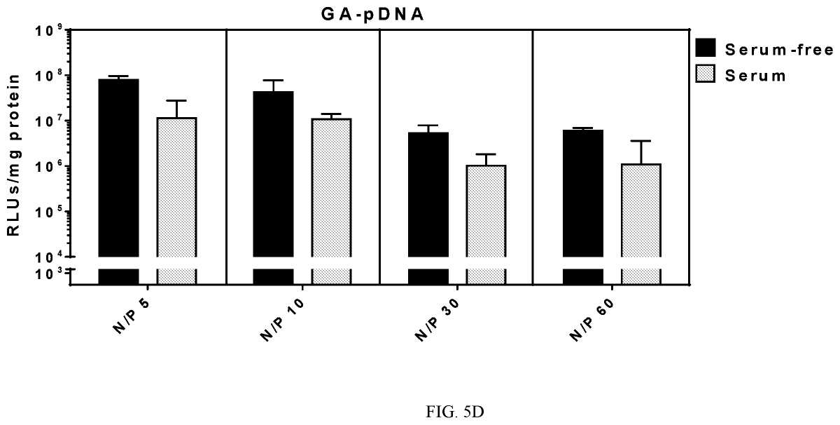

Generally, at the N/P ratios of 5 and 10, the transfection efficiency of the GA-pDNA nanoparticles without calcium were significantly higher than with calcium. Interestingly, the transfection efficiency of the K₁₀₀-pDNA nanoparticles were low at all N/P ratios. FIGS. 5A-B provide a comparision of the transfection efficiency of the different N/P ratios without (FIG. 5A) or with calcium (FIG. 5B). In the absence of calcium, the transfection efficiency of GA-pDNA nanoparticles were significantly higher than polylysine-pDNA nanoparticles (K₁₀₀-pDNA and K₉-pDNA nanoparticles). In the presence of calcium, both GA-pDNA and K₉-pDNA nanoparticles were significantly higher than the K₁₀₀-pDNA nanoparticles.

A549 and HeLa cells were also transfected in the presence of 10% fetal bovine serum. FIG. 5C depicts the transfection efficiency of GA-pDNA nanoparticles in the absence (serum-free) and presence of 10% fetal bovine serum (serum) at N/P ratios of 5, 10, 30, and 60. A slight decrease in the transfection efficiency value was observed in the presence of the 10% fetal bovine serum. Finally, FIG. 5D shows the transfection efficiency of GA-pDNA nanoparticles in HeLa cells at N/P ratios of 5, 10, 30, and 60. Here, the transfection efficiency of GA-pDNA and PEI-pDNA nanoparticles was significantly higher than the free pDNA.

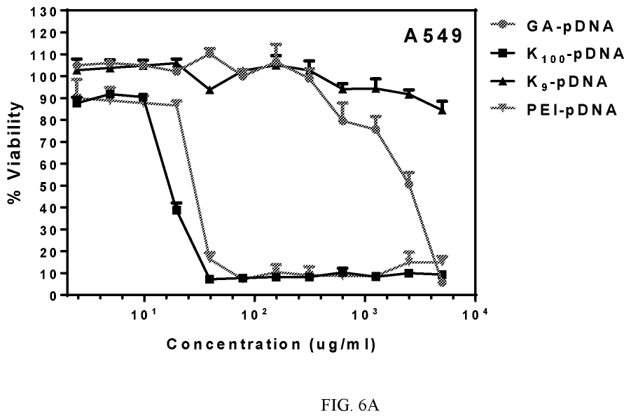

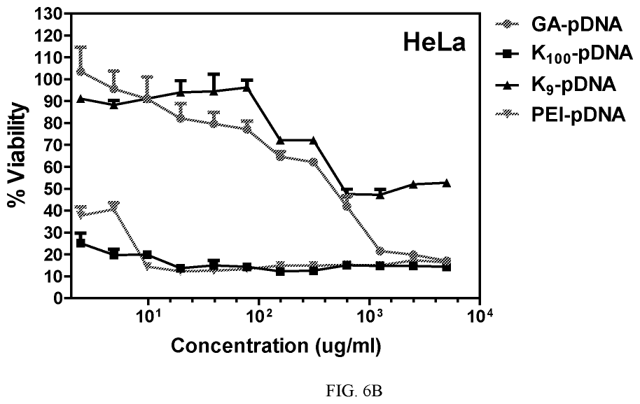

GA-pDNA nanoparticles exhibit low cytotoxicity. To examine whether GA-pDNA nanoparticles affected the viability of A549 and HeLa cells, an MTS cytotoxicity assay of GA, K₁₀₀, K₉, and PEI was conducted. The cytotoxicity profiles of GA, K₁₀₀, K₉, and PEI was determined in A549 cells (FIG. 6A) and HeLa cells (FIG. 6B). The figures show that K₁₀₀, K₁₀₀-pDNA nanoparticles, PEI, and PEI-pDNA nanoparticles are highly cytotoxic at low concentrations in both A549 cells and HeLa cell lines.

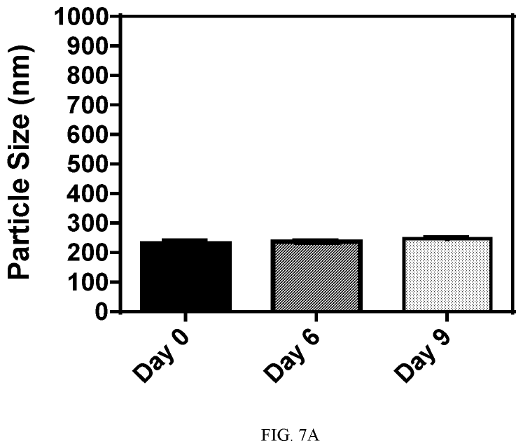

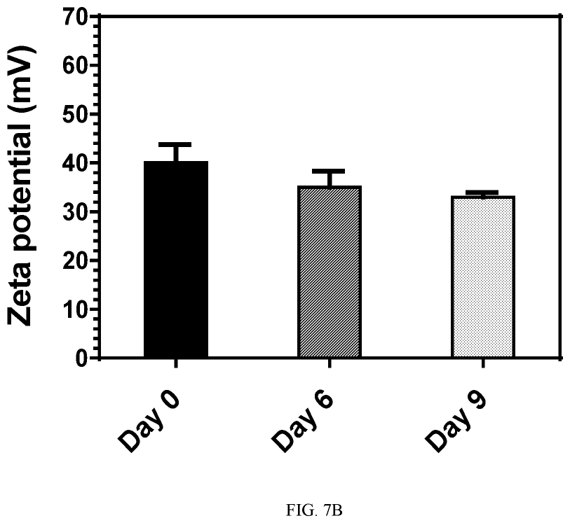

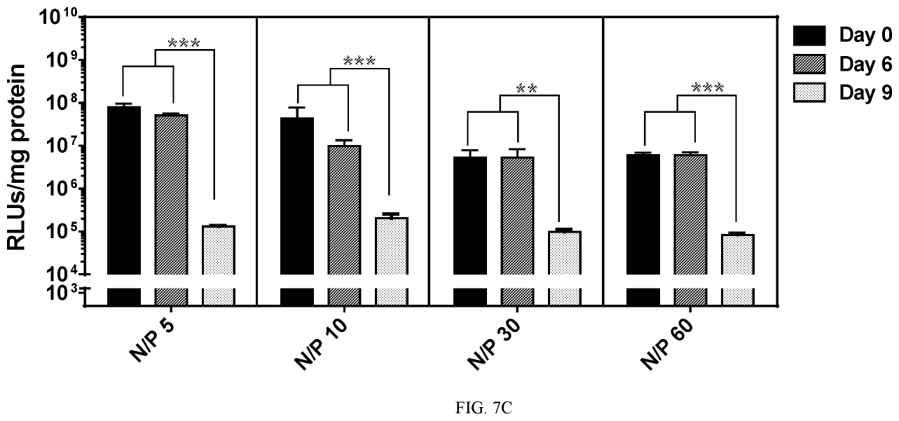

GA-pDNA nanoparticles are stable in solution for at least 6 days. The stability of GA-pDNA nanoparticles (N/P ratio of 10) stored at 4° C. was investigated. Particle size, zeta potential, and gene transfection efficiency of the nanoparticles were evaluated during the storage period. FIG. 7A shows the particle size of GA-pDNA nanoparticles at day 0, day 6, and day 9. There was no significant difference in the particle size over 9 days (−230 nm). FIG. 7B illustrates the zeta potential of GA-pDNA nanoparticles at for the same days. The stability studies showed a slight decrease in the zeta potential over 9 days (from 40 to 33 mV). Finally, FIG. 7C shows the transfection efficiency of GA-pDNA nanoparticles at N/P ratios of 5, 10, 30, and 60. The transfection efficiency between day 0 to day 6 was similar; however, the nanoparticles at day 9 were significantly less effective than at day 0 and day 6.

GA-CpG and GA-Poly(I:C) Complex Formation. Agarose gel electrophoresis studies can visually indicate complexation, or immobilization of the polyanion. Free Poly(I:C) or CpG runs freely through the agarose gel whereas GA does not. The agarose gels provided for analysis of the GA-CpG and GA-Poly(I:C) complexes with increasing GA in a mass ratio versus the polyanion counterpart. In the higher pH buffer, more GA (higher mass ratio) is required to fully immobilize the polyanion. The GA immobilizes Poly(I:C) at lower mass ratios than CpG, but this can be explained by the differences in molecular weight. At pH 7, Poly(I:C) appears to be fully immobilized at a mass ratio of 5:1 (mass ratio of 5:1=“R5”) whereas in pH 2 R2 is fully complexed. A similar trend was seen with CpG complexes where at pH 7 immobilization occurs at R10 and at pH 5 at R4. Complexes for this work were further made in 4% mannitol in water for injection.

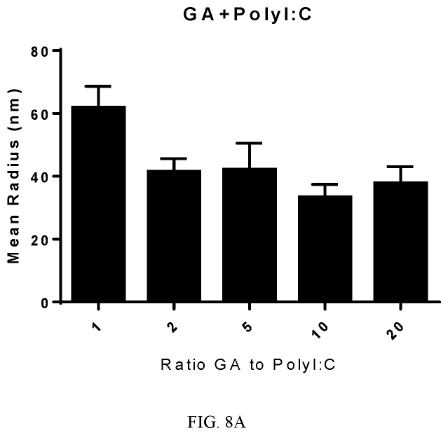

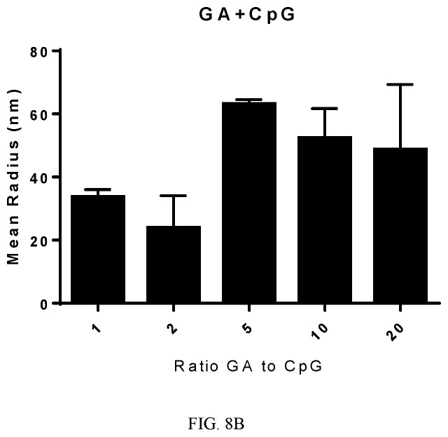

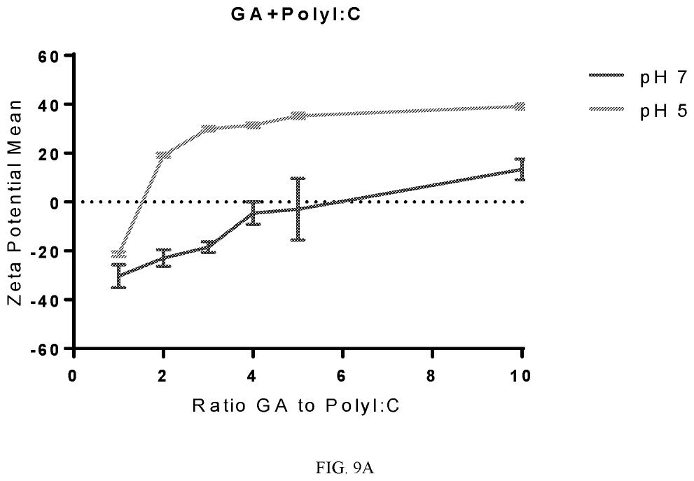

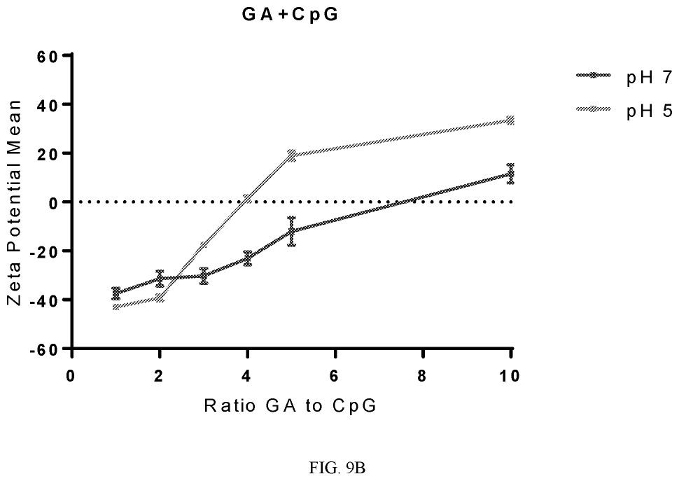

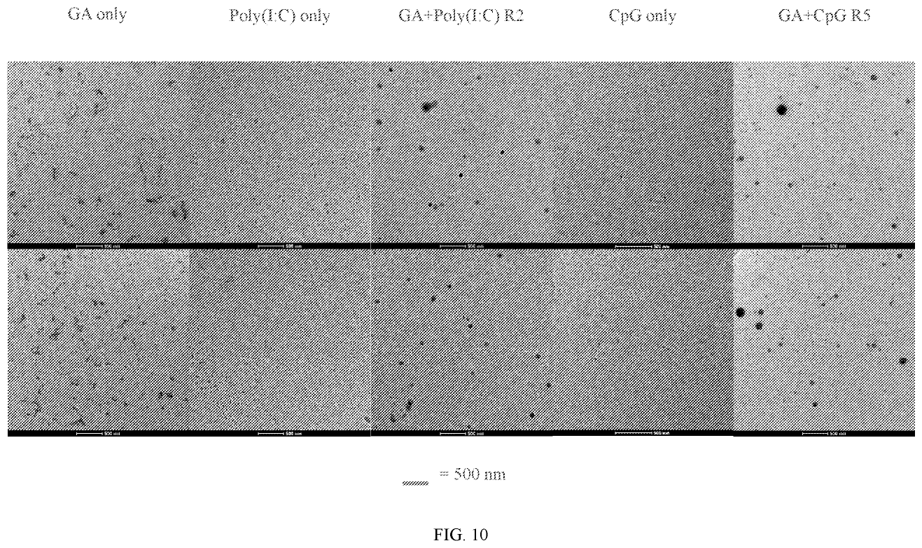

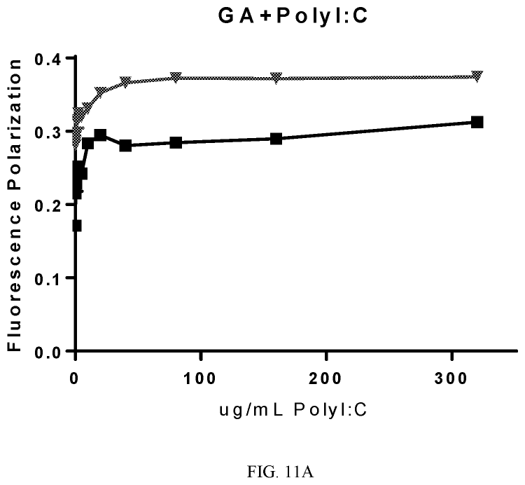

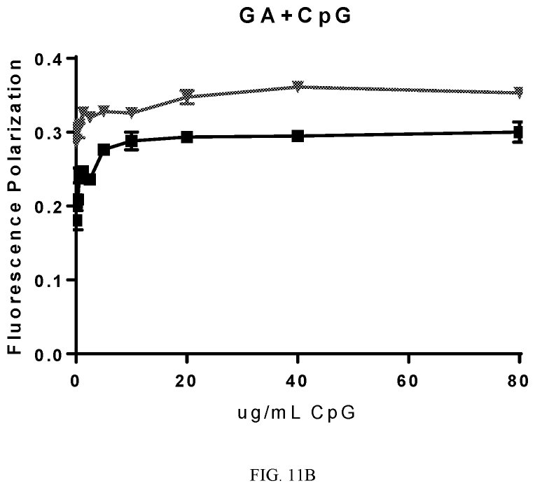

GA-CpG and GA-Poly(I:C) Complex Characterization. Zeta potential measurements (FIGS. 9A-B) neatly complemented the agarose gels showing a net positive charge around the same mass ratio that the gel indicates immobilization of the polyanion. The positive charge is important for potential cell uptake as it can increase the attractive force towards the negatively charged cell surface. At pH 7 both Poly(I:C) and CpG complexes require a higher GA ratio to achieve a net positive charge than at pH 5. At R1 the pH made less of an impact than at higher amounts of GA. At higher amounts of GA the charge starts to level off indicating an excess of GA. For all GA-CpG and GA-Poly(I:C) complexes, the radius of such particles fell between 20 and 70 nm (FIGS. 8A-B) as determined from DLS measurements—thus indicating particle diameters ranging from about 40 nm to about 140 nm. In addition, TEM images correlate with the range of particle sizes expected from the DLS measurements (FIG. 11).

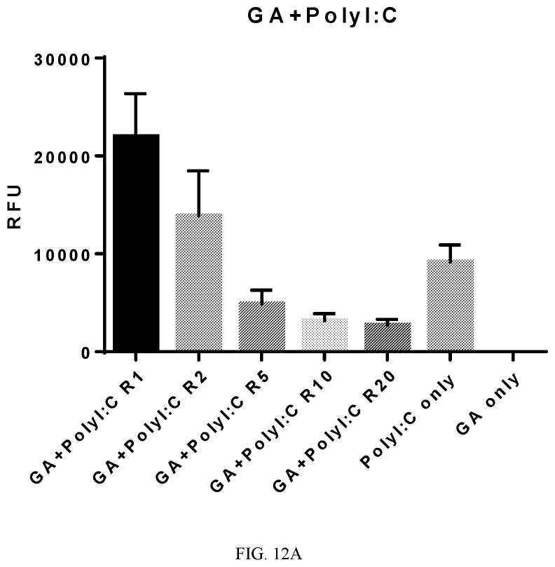

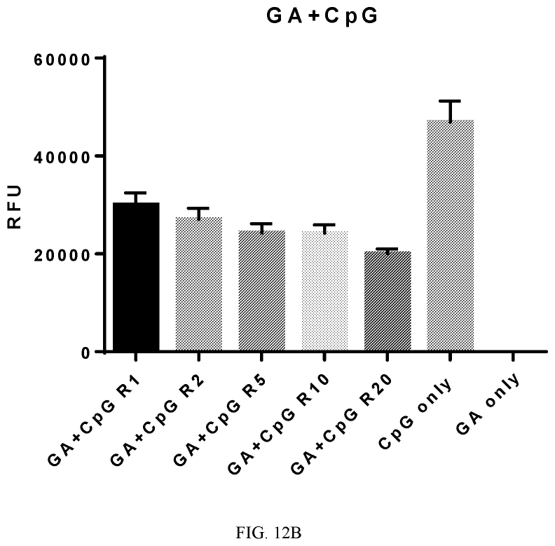

GA-CpG and GA-Poly(I:C) Complex Binding and Accessibility Characterization. Fluorescence polarization was utilized to monitor binding of fluorescently labeled-GA to CpG and Poly(I:C), where increase in polarization (“P”) indicates more immobilization. This alternative way to observe immobilization complements the agarose gel and zeta potential as the polarization increase levels off at the point in which the net charge is positive and where the gel indicates immobilization (FIGS. 12A-B).

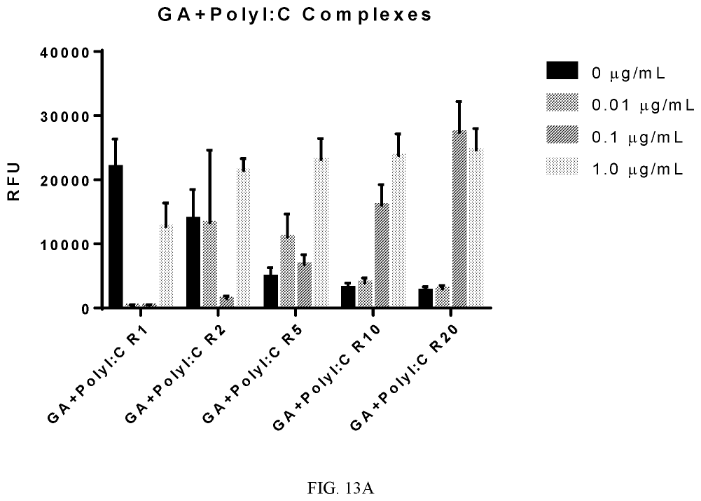

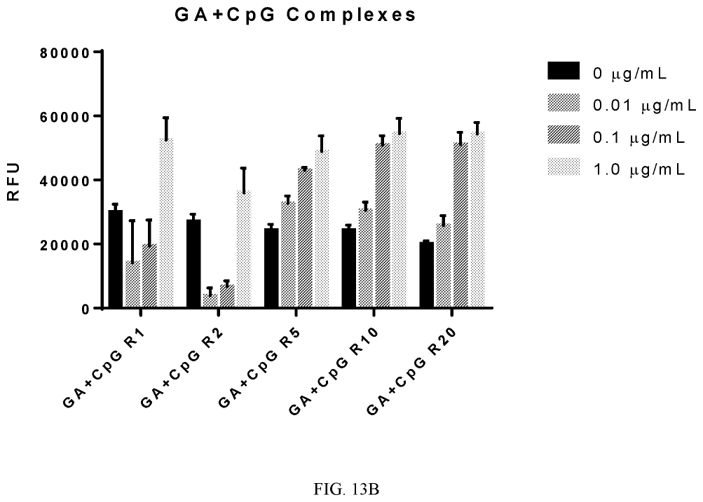

To further characterize the complex, accessibility of CpG and Poly(I:C) were assessed. FIGS. 12A-B shows the relative fluorescence after staining with SYBR Gold (which stains CpG and Poly(I:C)), illustrating decreasing fluorescence as GA is increased in the GA-CpG and GA-Poly(I:C) complexes and indicating that CpG and Poly(I:C) are becoming more encapsulated or complexed with increasing GA. Fluorescence measurements were also obtained after incubation with increasing amounts of dextran sulfate (FIGS. 13A-B).

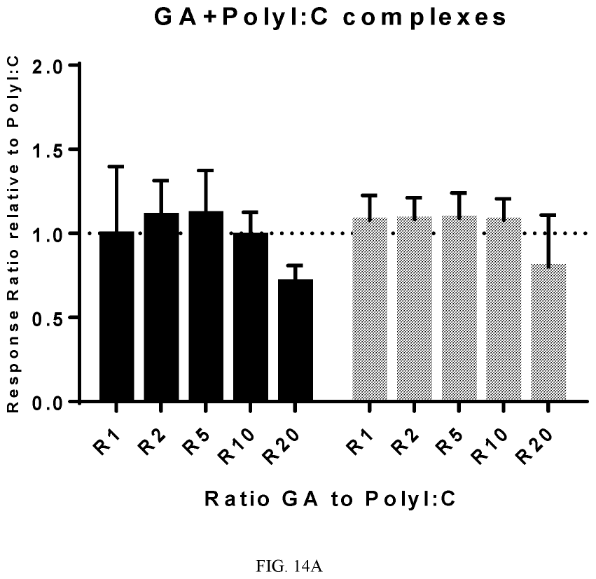

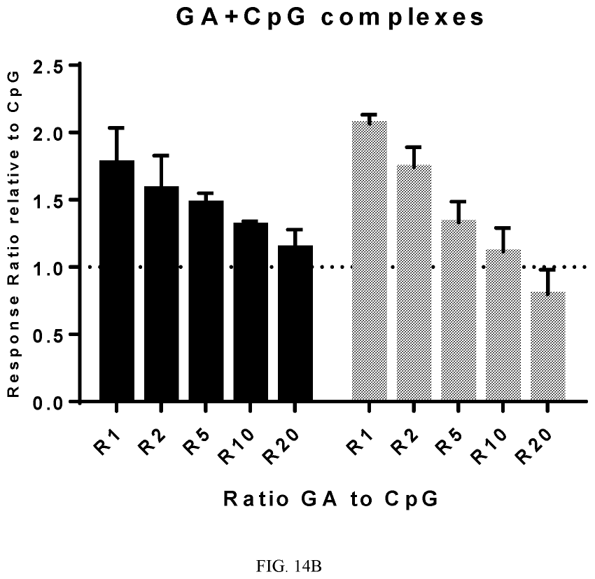

In vitro HEK blue reporter cell assay. Poly(I:C) and CpG alone are TLR agonists of TLR3 and TLR9, respectively, and stimulate immune response. HEK blue hTLR reporter cells were used to examine the effect of complexation on TLR activation. Poly(I:C) complexes and controls were run with TLR3 reporter cell line and CpG complexes and controls were run with TLR9. Samples were run in the Null cell line as an additional control. FIGS. 14A-B graph the absorbance of complex sample normalized to control, such that a value above 1 indicates activation of the TLR by the complex greater than by non-complexed Poly(I:C) (FIG. 14A) or CpG (FIG. 14B).

### Discussion Regarding GA-pDNA Nanoparticles

Lysine-rich polypeptides are a well-recognized non-viral gene vector and were one of the first polycations studied for complexation and delivery of genetic material.^{40, 43} The properties of K₉-pDNA (low molecular weight polylysine) and K₁₀₀-pDNA (high molecular weight polylysine) nanoparticles were compared to GA-pDNA nanoparticles. Prior studies have shown relatively low molecular weight polycations (e.g. ˜20,000 Da or less) complexed with pDNA exhibit smaller particle size and higher transfection efficiency when calcium is added as a condensing agent.¹ Prior studies also indicated that a final concentration in the range of 35-40 mmol/L CaCl₂ was optimal.^{7, 14, 37, 44}

Researchers investigated different lengths of lysine-rich CPPs to determine the optimal polylysine chain length (from three to 36 lysine residues) for genetic material condensation and transfection efficiency.⁴⁵ Adding CaCl₂ to K₉-pDNA nanoparticles decreased the particle size values, which is in good agreement with our previous studies.^{7, 14, 37, 38, 44} Moreover, calcium ion-dependent increase of the positive zeta potential of the K₉-pDNA nanoparticles may also play a significant role in enhancing the gene expression by the stronger ionic interaction with the negatively charged plasma membrane.⁴⁶ In the absence of CaCl₂, no significant level of transfection efficiency for the K₉-pDNA nanoparticles were detected. However, in the case of high-molecular-weight CPPs (e.g., K₁₀₀), the presence of CaCl₂ does not change the low gene expression of the K₁₀₀-pDNA nanoparticles. The gene expression of pDNA-Ca²⁺ complexes has been reported to be significantly lower than CPP-pDNA-Ca²⁺ complexes suggesting that CPPs in the nanoparticles is indeed significant to achieve the high transfection efficiency.^{7, 44}

Although there was no significant difference in the transfection efficiency between the K₁₀₀-pDNA and K₉-pDNA nanoparticles (in the absence of CaCl₂), the level of cytotoxicity of the K₁₀₀-pDNA nanoparticles was significantly higher than that of the K₉-pDNA nanoparticles. However, K₁₀₀ can bind to pDNA tighter and form smaller, higher positively charged, and more stable nanoparticle than K₉ and GA to the greater density and abundance of positive charge.^{47,48} Investigators highlighted the importance of the genetic material being released form the polyplexes to function. Unpackaging and release remain concerns with genetic vectors formed through electrostatic (ionic) bound with vectors.^{1, 7}

Surprisingly, the inventors of the present technology identified N/P ratios where GA was an effective transfection agent, without the need for added calcium. In addition, GA-pDNA were simple to formulate, were stable for several days, and yielded negligible cytotoxicity. GA achieved these desirable attributes as a gene vector by complexing with negatively charged pDNA to produce small, stable, and highly positively charged nanoparticles. Without being bound by theory, it is believed the positively-charged lysine residues of GA play a key role binding to proteins on the cell membrane to facilitate uptake by target cells.⁴⁹⁻⁵²

Polycations designed for gene delivery often consist of amphiphilic or cationic sequences of ˜30 residues, and they are particularly promising to deliver genetic material.^{1, 38, 53} Numerous physiochemical properties of polycations (e.g., charge, stability, and molecular weight) can alter the transfection efficiency of synthetic non-viral gene vectors.⁷ Peptides having a continuous non-polar domain (e.g., alanine, tyrosine, and tryptophan) can form stable complexes with pDNA and also efficiently deliver the pDNA into target cells.⁵⁴ For example, the relative balance of hydrophobic domains and positively charged domains are very important for membrane penetration of cell-penetrating peptides (CPPs).⁵⁵ Upon the interaction of peptides with cellular membranes, the positively charged cluster at the lipid-peptides interface establishing strong ionic (electrostatic) interactions with the negatively charged phospholipid cell membranes. The hydrophobic face of the peptides will interact and insert into the cell membranes through hydrophobic interactions, and cause an increase in the penetration potential (membrane perturbation).^{56, 57} The amphiphilic or hydrophobicity of peptides enhances their uptake into the cytoplasm, the cytoplasmic release, and their scape rate from endosomes.^{58, 59} Thus, the other hydrophobic amino acids (i.e., alanine and tyrosine) of GA could contribute to the high transfection efficiency we observed.

Furthermore, glutamic acid residues may augment the cellular uptake of CPPs.⁶⁰ GALA is a synthetic amphipathic CPP (fusogenic CPP) that contains glutamic acid, which is soluble at pH 7.5 and destabilizes membrane bilayers at a pH less than 6.0. At acidic pH, the protonation of the glutamic acid of GALA peptide destabilized endosomal/lysosomal membranes and promoted endosomal escape.^{1, 61} Moreover, adding polylysine to HA-2 peptide (GLF GAI AGFI ENGW EGMI DGWYG (SEQ ID NO: 3)) improved the endosomal release of the HA-2 peptide.^{35, 62} Accordingly, the negatively charged amino acid (i.e., glutamic acid) of GA may play a role in the transfection mechanism of GA-pDNA nanoparticles. Without being bound by theory, the cationic, anionic, and hydrophobic amino acid residues of GA may collectively condense large genetic material (e.g., pDNA), enhance transfection efficiency, facilitate the endosomal escape, and ensure the cytosolic delivery and release of genetic material.

### REFERENCES

1. Alhakamy, N. A.; Nigatu, A. S.; Berkland, C. J.; Ramsey, J. D. Noncovalently associated cell-penetrating peptides for gene delivery applications. Therapeutic delivery 2013, 4, (6), 741-757.
2. Yin, H.; Kanasty, R. L.; Eltoukhy, A. A.; Vegas, A. J.; Dorkin, J. R.; Anderson, D. G. Non-viral vectors for gene-based therapy. Nature Reviews Genetics 2014, 15, (8), 541-555.
3. Ginn, S. L.; Alexander, I. E.; Edelstein, M. L.; Abedi, M. R.; Wixon, J. Gene therapy clinical trials worldwide to 2012—an update. The journal of gene medicine 2013, 15, (2), 65-77.
4. Heitz, F.; Morris, M. C.; Divita, G. Twenty years of cell-penetrating peptides: from molecular mechanisms to therapeutics. British journal of pharmacology 2009, 157, (2), 195-206.
5. Han, S.-o.; Mahato, R. I.; Sung, Y. K.; Kim, S. W. Development of biomaterials for gene therapy. Molecular Therapy 2000, 2, (4), 302-317.
6. Lindgren, M.; Rosenthal-Aizman, K.; Saar, K.; Eiríksdóttir, E.; Jiang, Y.; Sassian, M.; Östlund, P.; Hällbrink, M.; Langel, Ü. Overcoming methotrexate resistance in breast cancer tumour cells by the use of a new cell-penetrating peptide. Biochemical pharmacology 2006, 71, (4), 416-425.
7. Alhakamy, N. A.; Dhar, P.; Berkland, C. J. Charge type, charge spacing, and hydrophobicity of arginine-rich cell-penetrating peptides dictate gene transfection. Molecular pharmaceutics 2016, 13, (3), 1047-1057.
8. Margus, H.; Padari, K.; Pooga, M. Cell-penetrating peptides as versatile vehicles for oligonucleotide delivery. Molecular Therapy 2012, 20, (3), 525-533.
9. Zuhorn, I. S.; Engberts, J. B.; Hoekstra, D. Gene delivery by cationic lipid vectors: overcoming cellular barriers. European Biophysics Journal 2007, 36, (4-5), 349-362.
10. Nakamura, Y.; Kogure, K.; Futaki, S.; Harashima, H. Octaarginine-modified multifunctional envelope-type nano device for siRNA. Journal of controlled release 2007, 119, (3), 360-367.
11. Khondee, S.; Baoum, A.; Siahaan, T. J.; Berkland, C. Calcium condensed LABL-TAT complexes effectively target gene delivery to ICAM-1 expressing cells. Molecular pharmaceutics 2011, 8, (3), 788-798.
12. Chen, S.; Han, K.; Yang, J.; Lei, Q.; Zhuo, R.-X.; Zhang, X.-Z. Bioreducible polypeptide containing cell-penetrating sequence for efficient gene delivery. Pharmaceutical research 2013, 30, (8), 1968-1978.
13. Kay, M. A.; Glorioso, J. C.; Naldini, L. Viral vectors for gene therapy: the art of turning infectious agents into vehicles of therapeutics. Nature medicine 2001, 7, (1), 33-40.
14. Alhakamy, N. A.; Berkland, C. J. Polyarginine molecular weight determines transfection efficiency of calcium condensed complexes. Molecular pharmaceutics 2013, 10, (5), 1940-1948.
15. Baum, C.; Kustikova, O.; Modlich, U.; Li, Z.; Fehse, B. Mutagenesis and oncogenesis by chromosomal insertion of gene transfer vectors. Human gene therapy 2006, 17, (3), 253-263.
16. Bessis, N.; GarciaCozar, F.; Boissier, M. Immune responses to gene therapy vectors: influence on vector function and effector mechanisms. Gene therapy 2004, 11, S10-S17.
17. Waehler, R.; Russell, S. J.; Curiel, D. T. Engineering targeted viral vectors for gene therapy. Nature Reviews Genetics 2007, 8, (8), 573-587.
18. Pack, D. W.; Hoffman, A. S.; Pun, S.; Stayton, P. S. Design and development of polymers for gene delivery. Nature reviews Drug discovery 2005, 4, (7), 581-593.
19. Mintzer, M. A.; Simanek, E. E. Nonviral vectors for gene delivery. Chemical reviews 2008, 109, (2), 259-302.
20. Jalilian, B.; Einarsson, H. B.; Vorup-Jensen, T. Glatiramer acetate in treatment of multiple sclerosis: a toolbox of random co-polymers for targeting inflammatory mechanisms of both the innate and adaptive immune system? International journal of molecular sciences 2012, 13, (11), 14579-14605.
21. Sela, M. The concept of specific immune treatment against autoimmune diseases. International reviews of immunology 1999, 18, (3), 201-216.
22. Teitelbaum, D.; Meshorer, A.; Hirshfeld, T.; Arnon, R.; Sela, M. Suppression of experimental allergic encephalomyelitis by a synthetic polypeptide. European journal of immunology 1971, 1, (4), 242-248.
23. Ben-Nun, A.; Kaushansky, N.; Kawakami, N.; Krishnamoorthy, G.; Berer, K.; Liblau, R.; Hohlfeld, R.; Wekerle, H. From classic to spontaneous and humanized models of multiple sclerosis: impact on understanding pathogenesis and drug development. Journal of autoimmunity 2014, 54, 33-50.
24. Aharoni, R. The mechanism of action of glatiramer acetate in multiple sclerosis and beyond. Autoimmunity reviews 2013, 12, (5), 543-553.
25. Aharoni, R. Immunomodulation neuroprotection and remyelination—the fundamental therapeutic effects of glatiramer acetate: a critical review. Journal of autoimmunity 2014, 54, 81-92.
26. Lalive, P. H.; Neuhaus, O.; Benkhoucha, M.; Burger, D.; Hohlfeld, R.; Zamvil, S. S.; Weber, M. S. Glatiramer acetate in the treatment of multiple sclerosis. CNS drugs 2011, 25, (5), 401-414.
27. Varkony, H.; Weinstein, V.; Klinger, E.; Sterling, J.; Cooperman, H.; Komlosh, T.;

Ladkani, D.; Schwartz, R. The glatiramoid class of immunomodulator drugs. Expert opinion on pharmacotherapy 2009, 10, (4), 657-668.
28. Putnam, D. Polymers for gene delivery across length scales. Nature materials 2006, 5, (6), 439-451.
29. de Raad, M.; Teunissen, E. A.; Lelieveld, D.; Egan, D. A.; Mastrobattista, E. High-content screening of peptide-based non-viral gene delivery systems. Journal of controlled release 2012, 158, (3), 433-442.
30. Alhakamy, N. A.; Kaviratna, A.; Berkland, C. J.; Dhar, P. Dynamic measurements of membrane insertion potential of synthetic cell penetrating peptides. Langmuir 2013, 29, (49), 15336-15349.
31. Kwoh, D. Y.; Coffin, C. C.; Lollo, C. P.; Jovenal, J.; Banaszczyk, M. G.; Mullen, P.; Phillips, A.; Amini, A.; Fabrycki, J.; Bartholomew, R. M. Stabilization of poly-L-lysine/DNA polyplexes for in vivo gene delivery to the liver. Biochimica et Biophysica Acta (BBA)—Gene Structure and Expression 1999, 1444, (2), 171-190.
32. Ward, C. M.; Read, M. L.; Seymour, L. W. Systemic circulation of poly (L-lysine)/DNA vectors is influenced by polycation molecular weight and type of DNA: differential circulation in mice and rats and the implications for human gene therapy. Blood 2001, 97, (8), 2221-2229.
33. Xu, B.; Wiehle, S.; Roth, J.; Cristiano, R. The contribution of poly-L-lysine, epidermal growth factor and streptavidin to EGF/PLL/DNA polyplex formation. Gene therapy 1998, 5, (9).
34. Wagner, E.; Cotten, M.; Foisner, R.; Birnstiel, M. L. Transferrin-polycation-DNA complexes: the effect of polycations on the structure of the complex and DNA delivery to cells. Proceedings of the National Academy of Sciences 1991, 88, (10), 4255-4259.
35. Wagner, E.; Plank, C.; Zatloukal, K.; Cotten, M.; Birnstiel, M. L. Influenza virus hemagglutinin HA-2 N-terminal fusogenic peptides augment gene transfer by transferrin-polylysine-DNA complexes: toward a synthetic virus-like gene-transfer vehicle. Proceedings of the National Academy of Sciences 1992, 89, (17), 7934-7938.
36. Stankovics, J.; Crane, A. M.; Andrews, E.; Wu, C. H.; Wu, G. Y.; Ledley, F. D.

Overexpression of human methylmalonyl CoA mutase in mice after in vivo gene transfer with asialoglycoprotein/polylysine/DNA complexes. Human gene therapy 1994, 5, (9), 1095-1104.
37. Alhakamy, N. A.; Ishiguro, S.; Uppalapati, D.; Berkland, C. J.; Tamura, M. AT2R gene delivered by condensed polylysine complexes attenuates Lewis lung carcinoma after intravenous injection or intratracheal spray. Molecular cancer therapeutics 2016, 15, (1), 209-218.
38. Baoum, A.; Xie, S.-X.; Fakhari, A.; Berkland, C. “Soft” calcium crosslinks enable highly efficient gene transfection using TAT peptide. Pharmaceutical research 2009, 26, (12), 2619-2629.
39. Baoum, A. A.; Berkland, C. Calcium condensation of DNA complexed with cell-penetrating peptides offers efficient, noncytotoxic gene delivery. Journal of pharmaceutical sciences 2011, 100, (5), 1637-1642.
40. McKenzie, D. L.; Collard, W. T.; Rice, K. G. Comparative gene transfer efficiency of low molecular weight polylysine DNA-condensing peptides. Chemical Biology & Drug Design 1999, 54, (4), 311-318.
41. Gottschalk, S.; Sparrow, J.; Hauer, J.; Mims, M.; Leland, F.; Woo, S.; Smith, L. A novel DNA-peptide complex for efficient gene transfer and expression in mammalian cells. Gene therapy 1996, 3, (5), 448-457.
42. Wadhwa, M. S.; Collard, W. T.; Adami, R. C.; McKenzie, D. L.; Rice, K. G. Peptide-mediated gene delivery: influence of peptide structure on gene expression. Bioconjugate chemistry 1997, 8, (1), 81-88.
43. Mo, R. H.; Zaro, J. L.; Shen, W.-C. Comparison of cationic and amphipathic cell penetrating peptides for siRNA delivery and efficacy. Molecular pharmaceutics 2011, 9, (2), 299-309.
44. Ishiguro, S.; Alhakamy, N. A.; Uppalapati, D.; Delzeit, J.; Berkland, C. J.; Tamura, M.

Combined Local Pulmonary and Systemic Delivery of AT2R Gene by Modified TAT Peptide Nanoparticles Attenuates Both Murine and Human Lung Carcinoma Xenografts in Mice. Journal of pharmaceutical sciences 2017, 106, (1), 385-394.
45. Kim, W. J.; Christensen, L. V.; Jo, S.; Yockman, J. W.; Jeong, J. H.; Kim, Y.-H.; Kim,

S. W. Cholesteryl oligoarginine delivering vascular endothelial growth factor siRNA effectively inhibits tumor growth in colon adenocarcinoma. Molecular Therapy 2006, 14, (3), 343-350.
46. Nam, H. Y.; Kim, J.; Kim, S.; Yockman, J. W.; Kim, S. W.; Bull, D. A. Cell penetrating peptide conjugated bioreducible polymer for siRNA delivery. Biomaterials 2011, 32, (22), 5213-5222.
47. Mitchell, D. J.; Steinman, L.; Kim, D.; Fathman, C.; Rothbard, J. Polyarginine enters cells more efficiently than other polycationic homopolymers. Chemical Biology & Drug Design 2000, 56, (5), 318-325.
48. Kim, H. H.; Choi, H. S.; Yang, J. M.; Shin, S. Characterization of gene delivery in vitro and in vivo by the arginine peptide system. International journal of pharmaceutics 2007, 335, (1), 70-78.
49. Kawabata, A.; Baoum, A.; Ohta, N.; Jacquez, S.; Seo, G.-M.; Berkland, C.; Tamura, M. Intratracheal administration of a nanoparticle-based therapy with the angiotensin II type 2 receptor gene attenuates lung cancer growth. Cancer research 2012, 72, (8), 2057-2067.
50. Felgner, P. L.; Gadek, T. R.; Holm, M.; Roman, R.; Chan, H. W.; Wenz, M.; Northrop, J. P.; Ringold, G. M.; Danielsen, M. Lipofection: a highly efficient, lipid-mediated DNA-transfection procedure. Proceedings of the National Academy of Sciences 1987, 84, (21), 7413-7417.
51. Hofland, H. E.; Nagy, D.; Liu, J.-J.; Spratt, K.; Lee, Y.-L.; Danos, O.; Sullivan, S. M. In vivo gene transfer by intravenous administration of stable cationic lipid/DNA complex. Pharmaceutical research 1997, 14, (6), 742-749.
52. Dennison, S. R.; Phoenix, A. J.; Phoenix, D. A. Effect of salt on the interaction of Hal18 with lipid membranes. European Biophysics Journal 2012, 41, (9), 769-776.
53. van Asbeck, A. H.; Beyerle, A.; McNeill, H.; Bovee-Geurts, P. H.; Lindberg, S.; Verdurmen, W. P.; Hällbrink, M.; Langel, U. l.; Heidenreich, O.; Brock, R. Molecular parameters of siRNA-cell penetrating peptide nanocomplexes for efficient cellular delivery. ACS nano 2013, 7, (5), 3797-3807.
54. Niidome, T.; Ohmori, N.; Ichinose, A.; Wada, A.; Mihara, H.; Hirayama, T.; Aoyagi, H. Binding of cationic α-helical peptides to plasmid DNA and their gene transfer abilities into cells. Journal of Biological Chemistry 1997, 272, (24), 15307-15312.
55. Shai, Y.; Oren, Z. Diastereomers of cytolysins, a novel class of potent antibacterial peptides. Journal of Biological Chemistry 1996, 271, (13), 7305-7308.
56. Teixeira, V.; Feio, M. J.; Bastos, M. Role of lipids in the interaction of antimicrobial peptides with membranes. Progress in lipid research 2012, 51, (2), 149-177.
57. Bechara, C.; Sagan, S. Cell-penetrating peptides: 20 years later, where do we stand? FEBS letters 2013, 587, (12), 1693-1702.
58. Gerke, V.; Moss, S. E. Annexins: from structure to function. Physiological reviews 2002, 82, (2), 331-371.
59. LeDoan, T.; Etore, F.; Tenu, J.-P.; Letourneux, Y.; Agrawal, S. Cell binding, uptake and cytosolic partition of HIV anti-gag phosphodiester oligonucleotides 3′-linked to cholesterol derivatives in macrophages. Bioorganic & medicinal chemistry 1999, 7, (11), 2263-2269.
60. Sun, C.; Shen, W.-C.; Tu, J.; Zaro, J. L. Interaction between cell-penetrating peptides and acid-sensitive anionic oligopeptides as a model for the design of targeted drug carriers. Molecular pharmaceutics 2014, 11, (5), 1583-1590.
61. Varkouhi, A. K.; Scholte, M.; Storm, G.; Haisma, H. J. Endosomal escape pathways for delivery of biologicals. Journal of Controlled Release 2011, 151, (3), 220-228.
62. Plank, C.; Oberhauser, B.; Mechtler, K.; Koch, C.; Wagner, E. The influence of endosome-disruptive peptides on gene transfer using synthetic virus-like gene transfer systems. Journal of Biological Chemistry 1994, 269, (17), 12918-12924.
63. Abdallah, B.; Hassan, A.; Benoist, C.; Goula, D.; Behr, J. P.; Demeneix, B. A. A powerful nonviral vector for in vivo gene transfer into the adult mammalian brain: polyethylenimine. Human gene therapy 1996, 7, (16), 1947-1954.
64. Boussif, O.; Lezoualc'h, F.; Zanta, M. A.; Mergny, M. D.; Scherman, D.; Demeneix, B.; Behr, J.-P. A versatile vector for gene and oligonucleotide transfer into cells in culture and in vivo: polyethylenimine. Proceedings of the National Academy of Sciences 1995, 92, (16), 7297-7301.
65. Huang, C. Y.; Ma, S. S.; Lee, S.; Radhakrishnan, R.; Braun, C. S.; Choosakoonkriang, S.; Wiethoff, C. M.; Lobo, B. A.; Middaugh, C. R. Enhancements in gene expression by the choice of plasmid DNA formulations containing neutral polymeric excipients. Journal of pharmaceutical sciences 2002, 91, (5), 1371-1381.
66. Wiethoff, C. M.; Koe, J. G.; Koe, G. S.; Middaugh, C. R. Compositional effects of cationic lipid/DNA delivery systems on transgene expression in cell culture. Journal of pharmaceutical sciences 2004, 93, (1), 108-123.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A composition comprising a plurality of nanoparticles, each nanoparticle of the plurality of nanoparticles comprising
   a glatiramoid; and
   one or more polynucleotides comprising a polyinosine-polycytidylic acid (Poly(I:C)), a plasmid DNA (pDNA), a CpG oligodeoxynucleotide (CpG ODN), or a combination of any two or more thereof.
B. The composition of Paragraph A, wherein the nanoparticles are configured to possess a ratio of cationic charges of the glatiramoid (N) to phosphate anionic charges of polynucleotide (P) of about 0.5:1 to about 100:1.
C. The composition of Paragraph A or Paragraph B, wherein the nanoparticles are configured to possess a mass ratio of glatiramoid to polynucleotide of about 0.5:1 to about 30:1.
D. The composition of any one of Paragraphs A-C, wherein the Poly(I:C) has a weight average number of base pairs of about 0.2 kb to about 8 kb.
E. The composition of any one of Paragraphs A-D, wherein the CpG ODN comprises a Class A CpG ODN, a Class B CpG ODN, a Class C CpG ODN, or a combination of any two or more thereof.
F. The composition of any one of Paragraphs A-E, wherein the CpG ODN comprises Class B CpG ODN 1825, Class B CpG ODN 2006, Class B CpG ODN BW006, Class B CpG ODN 1668, Class A CpG ODN 1585, Class A CpG ODN 2216, Class A CpG ODN 2336, Class C CpG ODN 2395, Class C CpG ODN M362, or a combination of any two or more thereof.
G. The composition of any one of Paragraphs A-F, wherein the pDNA comprises angiotensin II type 2 receptor pDNA, pDNA encoding anti-HER2 antibody, pDNA encoding murine interferon α, or a combination of any two or more thereof.
H. The composition of any one of Paragraphs A-G, wherein the glatiramoid has a weight average molecular weight of about 5,000 to about 18,000.
I. The composition of any one of Paragraphs A-H, wherein the glatiramoid comprises glatiramer acetate (GA), protirmamer, or both.
J. The composition of any one of Paragraphs A-I, wherein the nanoparticles have an intensity-weighted average diameter as determined by dynamic light scattering of about 20 nm to about 500.
K. The composition of any one of Paragraphs A-J, wherein the composition further comprises a pharmaceutically acceptable carrier.
L. The composition of any one of Paragraphs A-K, wherein the composition further comprises water.
M. The composition of any one of Paragraphs A-L, wherein the composition is formulated for parenteral administration.
N. The composition of any one of Paragraphs A-M, wherein the composition is at a pH of about 5 to about 10.
O. The composition of any one of Paragraphs A-N, wherein the composition comprises CaCl₂ at a concentration no greater than about 1 mM.
P. The composition of any one of Paragraphs A-O, wherein the composition comprises CaCl₂ at a concentration no greater than about 1 nanomolar.
Q. A method for delivering a polyinosine-polycytidylic acid (Poly(I:C)), a plasmid DNA (pDNA), a CpG oligodeoxynucleotide (CpG ODN), or a combination of any two or more thereof, to a subject, the method comprising administering a composition of any one of Paragraphs A-P to the subject.

Other embodiments are set forth in the following claims, along with the full scope of equivalents to which such claims are entitled.

1. A composition comprising a plurality of nanoparticles, each nanoparticle of the plurality of nanoparticles comprising
a glatiramoid; and
one or more polynucleotides comprising a polyinosine-polycytidylic acid (Poly(I:C)), a plasmid DNA (pDNA), a CpG oligodeoxynucleotide (CpG ODN), or a combination of any two or more thereof

2. The composition of claim 1, wherein the nanoparticles are configured to possess a ratio of cationic charges of the glatiramoid (N) to phosphate anionic charges of polynucleotide (P) of about 0.5:1 to about 100:1.

3. The composition of claim 1, wherein the nanoparticles are configured to possess a mass ratio of glatiramoid to polynucleotide of about 0.5:1 to about 30:1.

4. The composition of claim 1, wherein the Poly(I:C) has a weight average number of base pairs of about 0.2 kb to about 8 kb.

5. The composition of claim 1, wherein the CpG ODN comprises a Class A CpG ODN, a Class B CpG ODN, a Class C CpG ODN, or a combination of any two or more thereof.

6. The composition of claim 1, wherein the CpG ODN comprises Class B CpG ODN 1825, Class B CpG ODN 2006, Class B CpG ODN BW006, Class B CpG ODN 1668, Class A CpG ODN 1585, Class A CpG ODN 2216, Class A CpG ODN 2336, Class C CpG ODN 2395, Class C CpG ODN M362, or a combination of any two or more thereof.

7. The composition of claim 1, wherein the pDNA comprises angiotensin II type 2 receptor pDNA, pDNA encoding anti-HER2 antibody, pDNA encoding murine interferon α, or a combination of any two or more thereof.

8. The composition of claim 1, wherein the glatiramoid has a weight average molecular weight of about 5,000 to about 18,000.

9. The composition of claim 1, wherein the glatiramoid comprises glatiramer acetate (GA), protirmamer, or both.

10. The composition of claim 1, wherein the nanoparticles have an intensity-weighted average diameter as determined by dynamic light scattering of about 20 nm to about 500.

11. The composition of claim 1, wherein the composition further comprises a pharmaceutically acceptable carrier.

12. The composition of claim 1, wherein the composition further comprises water.

13. The composition of claim 1, wherein the composition is formulated for parenteral administration.

14. The composition of claim 11, wherein the composition is at a pH of about 5 to about 10.

15. The composition of claim 14, wherein the composition comprises CaCl₂ at a concentration no greater than about 1 mM.

16. The composition of claim 14, wherein the composition comprises CaCl₂ at a concentration no greater than about 1 nanomolar.

17. A method for delivering a polyinosine-polycytidylic acid (Poly(I:C)), a plasmid DNA (pDNA), a CpG oligodeoxynucleotide (CpG ODN), or a combination of any two or more thereof, to a subject, the method comprising administering a composition of claim 1 to the subject.

18. The method of claim 17, wherein the nanoparticles are configured to possess a ratio of cationic charges of the glatiramoid (N) to phosphate anionic charges of polynucleotide (P) of about 0.5:1 to about 100:1.

19. The method of claim 17, wherein the nanoparticles are configured to possess a mass ratio of glatiramoid to polynucleotide of about 0.5:1 to about 30:1.

20. The method of claim 17, wherein the Poly(I:C) has a weight average number of base pairs of about 0.2 kb to about 8 kb.


SEQUENCE LISTING (3 sequences)

SEQ ID NO: 1 - protein, 9 aa, Artificial Sequence
  Description of Artificial Sequence Synthetic peptide
KKKKKKKKK

SEQ ID NO: 2 - protein, 100 aa, Artificial Sequence
  Description of Artificial Sequence Synthetic polypeptide
KKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKK

SEQ ID NO: 3 - protein, 23 aa, Influenza A virus
GLFGAIAGFIENGWEGMIDGWYG